(19) United States
(12) Patent Application Publication
(10) Pub. No.: US 2006/0015261 A1
(43) Pub. Date: Jan. 19, 2006

(54) Blood clotting predictor

(76) Inventors: Kenneth G. Mann, Grand Isle, VT (US); Stephen J. Everse, Shelburne, VT (US); Matthew F. Hockin, Salt Lake City, UT (US); Kenneth C. Jones, Montpeller, VT (US)

Correspondence Address: EDWARDS & ANGELL, LLP, P.O. BOX 55874, BOSTON, MA, 02205, US

(21) Appl. No.: 10/507,661

(22) PCT Filed: Mar. 10, 2003
(86) PCT No.: PCT/US03/07379

§ 371 Date: Aug. 11, 2005

Related U.S. Application Data
(60) Provisional application No. 60/363,223, filed on Mar. 11, 2002.

(51) Int. Cl. G06F 19/00; G01N 33/48; G01N 33/50

(52) U.S. Cl. 702/19

(57) ABSTRACT

A computer program product for predicting the speed and efficacy of a blood-clotting agent is disclosed. The product comprises a computer usable medium having computer readable program code means embodied in the medium for causing an application program to execute on a computer with a database for storing data therein. The computer readable program code means comprises a first computer readable program code means for causing the computer to enter data into the database from a user interface, a second computer readable program code means for causing the computer to enter chemical equations into the database according to a user's input, a third computer readable program code means for causing the computer to compile differential equations corresponding to the chemical equations, a fourth computer readable program code means for causing the computer to solve the differential equations, and a fifth computer readable program code means for causing the computer to display the results of the solution to the differential equations.

### FIELD OF THE INVENTION

This invention relates to blood coagulation, and more particularly, to a method and apparatus for predicting the efficacy and speed of a blood clotting reaction mixture.

### BACKGROUND OF THE INVENTION

Treatments for various diseases may require promoters, called procoagulants, or inhibitors, called anticoagulants or “thinners”, of blood coagulation in order to accelerate or retard the coagulation of blood, respectively. Many of these agents have been identified through empirical experiments on both animals and humans. The empirical testing of these blood coagulation agents on either animals or humans, however, is often undesirable, as it can lead to unwanted suffering or death when the agent does not function as anticipated.

Scientists have identified many of the chemical processes involved in the coagulation and thrombolysis of blood, and mathematical equations have been written that describe these chemical reactions. Not all of the reactions involved in blood coagulation have been identified to date, so a complete mathematical model of blood coagulation, based upon a complete understanding of the blood chemistry involved, has not been possible. Also, with the large number of reactions, and the multiple fates for each enzyme and cofactor, it becomes exceedingly complex to intuit the specific outcome of an intervention using rough estimations; therefore, predicting whether an agent will “clot ” or “thin” blood, and if so, how long it will take to function without empirical evidence, remains difficult at best

Nevertheless, it would be extremely useful if there were an empirical, mathematical model that would predict whether a given agent would coagulate blood, and if so, how long it would take to function, so as to avoid the testing of potential clotting agents on either animals or humans. Such a model would have many utilities, even thought it did not completely reflect the chemistry of blood clotting. A first useful application would be to determine whether the assumptions used in describing the sequence of reactions leading to thrombin formation accurately reflect the laboratory evidence, especially data relating those results to coagulation processes in whole blood. In this way, the laboratory could design future experiments in such a way as to focus on the critical steps of thrombin formation and suppression of its amplification. A second useful application would be to utilize patient data to focus on interventions that would restore hemostasis. For example, data from patients with vascular injury, or patients with hemophilia types A or B, would be appropriate candidates.

### BRIEF DESCRIPTION OF THE DRAWINGS

The objects and features of the invention can be better understood with reference to the following detailed description and accompanying drawings.

FIG. 1 shows total thrombin generation (thrombin+meizothrombin) as a function of Tissue Factor (TF) concentration with (closed symbols) and without (open symbols) TFPI. The concentrations of TF illustrated are 25 pM (circles), 5 pM (squares) and 1 pM (diamonds). The filled symbols represent experiments conducted with 2.5 nM TFPI present

FIG. 2 shows active thrombin present as a function of time for a reaction initiated with 25 pM tissue factor. The reactions represented are no inhibitors (circles), AT-III only (diamonds), Tissue Factor Pathway Inhibitor (TEPI) only (triangles), both inhibitors present (squares).

FIG. 3 shows total thrombin as a function of time is represented for varying initiating TF concentrations: 25 pM (filled circles), 20 pM (open triangles), 15 pM (open circles), 10 pM (filled triangles), 5 pM (filled squares), 1 pM (filled diamonds).

FIG. 4 shows peak area of active thrombin (thrombin!seconds) is plotted vs. TF concentration. Total thrombin is represented by open squares; active thrombin is represented by filled squares.

FIG. 5A shows concentration of various metabolites as a function of time for the first 30 seconds of a reaction initiated by 5 pM TF. Represented are active thrombin (squares), active factor VIIIa (diamonds), active factor Va (circles) and active factor Xa (triangles).

FIG. 5B shows active thrombin (squares) and active factor VIIIa (diamonds) as a function of time in the first 30 seconds for reactions with factor Va present (filled symbols) or absent (open symbols).

FIG. 6, shows metabolite concentrations over the first I00 seconds of the reaction initiated with 5 pM TF. Represented are active thrombin (filled squares), active factor VIIIa (filled diamonds), active factor IXa (open squares), intrinsic factor Xase complex (open diamonds), factor Va (filled circles), active factor Xa (filled triangles), and prothrombinase (open circles).

FIG. 7 shows the concentrations of active thrombin (closed squares), active factor VIIa (filled diamonds) and extrinsic factor Xase (open diamonds) as a function of time for the first 100 seconds for a reaction initiated with 5 pM TF.

FIG. 8A shows the concentration of active thrombin (filled squares), active factor VIIa (filled diamonds) and extrinsic factor Xase (open diamonds) are plotted as a function of time over the entire course of the reaction (1200 seconds) initiated with 5 pM TF.

FIG. 8B shows metabolites are plotted as a function of time over the entire (1200) course for the reaction initiated with 5 pM TF. Represented are active factor Xa (filled triangles), active factor Va (filled circles), prothrombinase (open circles), active factor IXa (open squares), active factor VIIIa (filled triangles) and intrinsic factor Xase complex (open diamonds).

FIG. 8C shows the concentration of factor Xa produced by the intrinsic factor Xase (filled triangles) and the extrinsic factor Xase (open triangles) is presented as a function of time. The insert to FIG. 8C illustrates the relative percentage of factor Xa produced by each catalyst.

FIG. 9 shows the concentrations of the inactivation products of the reaction are plotted over the entire 1200-second course for the reaction. Represented is the factor VIIIa-A₂ domain dissociation product (filled diamonds), factor Xa-AT-III complex (filled triangles), factor IXa-AT-III complex (open triangles), factor VIIa-TF-AT-III complex (filled circles) and the complex of all thrombin species with AT-III (filled squares).

FIG. 10A shows concentration of active thrombin as a function of time produced when the reaction is initiated with varying concentrations of prothrombin. Experimental conditions include 2.1 μM prothrombin (150%, filled diamonds); 1.75 μM prothrombin (125%, filled triangles); 1.4 μM prothrombin (100%, filled squares); 1.05 μM prothrombin (75%, filled circles); and 0.7 μM prothrombin (50%, asterisk).

FIG. 10B shows empirical data taken from the manuscript of Butenas et al. Active thrombin present as a function of time for 30 through 150% concentrations of prothrombin. See FIG. 10A for legend identification.

FIG. 10C shows a representation of the theoretical thrombin produced as a function of time under the experimental conditions of FIGS. 10A and 10B with initial conditions representing a combination of 95% factor V and 5% factor Va.

FIG. 11 is a block diagram representing the logic sequence for a software program in accord with the present invention.

FIGS. 12A to 12D are block diagrams representing the logic sequence for the solver program of FIG. 11.

FIG. 13 is a graphical window displayed to a user showing a login prompt for access to a modeling server.

FIG. 14 is a graphical window displayed to a user after acceptance of a password. Note the appearance of the left frame menu.

FIG. 15 is a graphical window displayed to a user showing a list of other users from which a user can select equations, species and/or rate constants.

FIG. 16 is a graphical window displayed to a user showing a list of stored equations generated from the selections of FIG. 15.

FIG. 17 is a graphical window displayed to a user showing a list of equations used in a run.

FIG. 18 is the lower half of the graphical window of FIG. 17 displayed to a user showing the lower half of the text box for the entry of equations wherein the user is also prompted to select initial species concentrations from prior data.

FIG. 19 is a graphical window displayed to a user allowing input of a species title and the initial species concentrations to be inserted into the database for a run.

FIG. 20 is the lower half of the graphical window of FIG. 20 wherein the user is prompted to select rate constants from prior data.

FIG. 21 is a graphical window displayed to a user allowing input of a rate constant title and rate constants to be inserted in to the database for a run.

FIG. 22 is the lower half of the graphical window of FIG. 21 wherein the user is prompted to enter rate constants for the equations in to the database.

FIG. 23 is a graphical window displayed to a user showing a text box for the entry of an experimental duration and the selection of a titration experiment.

FIG. 24 is a graphical window displayed to a user showing a pull down menu (in this example showing TF, but all species are available) and text boxes for the entry of initial species concentrations if the titration option was selected in FIG. 23.

FIG. 25 is a graphical window displayed to a user permitting the entry of a title to an experiment thus allowing future selection of this information in other experiments.

FIG. 26 is a graphical window displayed to a user showing a selection of data for a new graph.

FIG. 27 is the lower half of the graphical window of FIG. 26 wherein the user may select data for a new graph and an option to also select to view others users data for use in a comparison.

FIG. 28 is a graphical window displayed to a user showing a selection of output formats.

FIG. 29 is a graphical window displayed to a user showing a selection of image size for a graphical representation of data from a run.

FIG. 30 is a graphical window displayed to a user showing a graphical representation of data from a run as well as other users data that could be merged in.

FIG. 31 is a graphical window displayed to a user showing a selection of other users data (note checked boxes) to be compared.

### DETAILED DESCRIPTION OF THE PREFERRED EMBODIMENT

As discussed more fully hereinbelow, the, invention relates to a method and apparatus for modeling a molecular pathway and for predicting the effects of candidate compounds (e.g., drugs) on a plurality of steps in a cascade or pathway. Generally, as used herein, a “pathway ” (e.g., chemical reactions, binding reactions, and the like), such as those which are involved in biochemical, cellular, physiological, and/or pathophysiological processes. A pathway may interconnect with, and/or be regulated by one or more other pathways. Preferably, the one or more other pathways also can be modeled as a series of chemical reactions and/or binding reactions.

In one aspect, a pathway being modeled comprises “pathway components” (e.g., such as enzymes, substrates, cofactors, ligands, receptors, ions, signaling molecules, transport molecules, DNA, RNA, ribosomes, transcription factors, translation factors, and the like) and “interaction values” which are associated with the components (e.g., concentrations, rate constants, binding affinities, dissociation rates, catalysis rates, transfer rates, rates of synthesis, etc.) in a relational database according to the invention. Preferably, the invention provides a computer program product for manipulating the values to generate a series of time-dependent concentration profiles for any/all reactants/components in a pathway over any time frame of interest. More preferably, the computer program product implements a problem solver, such as a Runge-Kutta problem solver, to perform these computations.

The program product can be used to model the effects of additional objects introduced into the system (e.g., compounds, such as drugs, other pathway molecules, other pathways), the effects of modifications of existing components (e.g., protein modifications, mutations, etc.), and/or the effects of altering “system values” such as temperature, pH, and the like. In a preferred aspect, the program product models a pathway that is individualized for a particular patient, e.g., such as a patient suffering from a disease or predisposition to a disease.

In a particularly preferred embodiment, the invention is used to determine the efficacy and speed of a blood clotting agent on a blood coagulation pathway. Preferred practice involves using the invention to model the extrinsic coagulation system (particularly stoichiometric anticoagulants) and to accommodate the formation, expression, and propagation of the vitamin K dependent procoagulant complexes. In one embodiment, the model includes about 34 differential equations and about 42 rate constants. In this invention example, the invention can be employed to describe in about 27 independent equilibrium expressions, the fates of about 34 species.

The invention provides several important advantages. For example, preferred practice allows a user to analyze and manipulate blood clotting in silico. This feature of the invention provides further benefits. For example, it allows health provider to determine patient wellness at the level of blood coagulation. That is, by using the invention with patient-derived variables (rate constants and equilibrium expressions) it is possible to approximate in vivo blood clotting patterns. Reaction to particular diseases and therapies can now be predicted before onset of potentially life threatening blood clotting problems. Moreover, because the invention is flexile and designed to accommodate many blood coagulation variables simultaneously, the invention can be tailored to fit a particular patient or plurality of patients grouped by family and/or medical history, demographics, sex, age, etc. In this embodiment, the invention can be used to optimize therapies that impact to the blood coagulation cascade either directly or indirectly.

Further advantages are provided by the present invention. In particular, use of the invention will assist drug screens in which candidate compounds are known or suspected to impact blood coagulation. As an illustration, the invention can be used to confirm blood-clotting profiles in settings in which a particular candidate compound increases or decreases activity of one or more of the rate constants. Likely blood clotting outcomes can now be predicted while minimizing often costly and time-consuming wet chemical approaches. Candidate compounds which can be evaluated using the invention include, but are not limited to: small molecules; organic or inorganic compounds; proteins, polypeptides, peptides, or recombinant forms thereof; amino acids; nucleic acids; aptamers; ribozymes; antisense molecules; fibrinotides; nucleotides; metabolites; agonists; antagonists; and the like. Compounds may have a direct effect on a clotting pathway (e.g., by binding to a factor or clotting protein) or an indirect effect (e.g., a compound, such as a nucleic acid, may require expression by a cell prior to producing an effect). As referred to herein, “compounds” and “drugs” generally are used interchangeably to denote any agent with a known or potential bioactive effect on a molecular pathway being investigated, e.g., such as a blood coagulation pathway.

In one aspect, the invention provides a method for modeling the effect of a candidate compound on the blood coagulation pathway. For example, the effect of a compound, such as an inhibitor of clotting (e.g., an antithrombotic agent) or an accelerator of clotting (e.g., a therapeutic agent for treating a hemorrhagic disease) can be evaluated using a program according to the invention to identify particular step(s) of the pathway that would be affected by introduction of the compound. In this way, the program can be used to design an appropriate treatment strategy for a pathology which qualitatively or quantitatively alters steps of the pathway, i.e., providing a user of the program with the ability to select the parameters of a drug (e.g., range of binding affinities for a particular blood clotting factor, range of effects on catalysis, etc.) that would at least partially restore step(s) of the pathway to normal. Candidate compounds can then be screened to identify those that fit selected criteria defined by the model.

In one aspect, the invention provides a method for designing a compound with a selected pharmacological activity, i.e., a target and mechanisms for exploiting that target. The method comprises simulating a molecular pathway (e.g., such as a blood coagulation pathway) comprising a plurality of reaction steps and simulating a compound which interacts with one or more reactants/components of the pathway. Interaction parameters (e.g., binding affinities, antagonist or agonist activity, affects on catalysis, substrate association and disassociation rates, affects on other equilibrium constants, etc.) are identified which cause a desired effect on one or more steps of the pathway (to increase or decrease thrombin activity, for example). Compound features (e.g., physical, structural, chemical characteristics of the compound) are then identified which provide the selected activity. Identification of compound features provides a means to identify (e.g., screen for) and/or synthesize compounds that have the features and which, therefore, are likely to exhibit the desired pharmacological activity.

For example, the impact of a drug affecting one or more of the binding interactions shown in Table 1, below, can be modeled to identify a drug with a suitable window of pharmacological activity.

Preferably, a drug is selected which provides a desired effect on particular step(s) or target of the pathway over a relatively wide dose range of drug. In one aspect, the drug is a recombinant molecule, such as a recombinant protein, polypeptide, or peptide, which can produce an at least about 50-fold, 100-fold, 200-fold, greater than 1000-, or greater than about 10,000-fold increase or decrease in a blood coagulation pathway molecule (e.g., such as a molecule shown in Table 3).

In a preferred aspect, the program models a pathway that is individualized for a particular patient For example, patient-specific concentrations of pro- and anti-coagulants can be provided to a computer program product through a user interface communicating with a modeling server as described further below. Assuming that rate constants are unchanged, the computer program product can be used to generate time-dependent concentration profiles for reactants unique to that patient over a time frame of interest (e.g., during a period when symptoms are expressed or during a period when symptoms are not expressed or when the patient is exposed to a particular therapy). In one aspect, the patient has a congenital or acquired condition which affects blood clotting. Such conditions include, but are not limited to, deficiencies in one or more of fibrinogen, Factors II, V, VII, VIII, IX, X, XI and XII, and other Factors shown in Table 3, well deficiencies in ATIII, plasminogen, protein C, protein S. etc; and conditions caused by exposure to agents such as heparin, coumadin, etc. Changes in rate constants themselves also can be modeled by empirically determining rate constants unique to a patient using methods routine in the art.

By modeling individualized pathways, the invention provides a means of modeling a patient-specific reaction to a drug. Therefore, in one aspect, the effect of a known compound or a compound suspected of having an activity is evaluated using a patient-specific model of blood coagulation. Preferably, the patient has a condition associated with increased or decreased blood clotting compared to normal patients. More preferably, parameters of a compound are identified which would normalize steps, concentrations, and/or kinetics of the coagulation pathway. Identification of such parameters enables a user to rationally design a drug with specific therapeutic endpoints in mind.

Still further advantages are provided by the invention. For example, and in contrast to prior models, the invention provides a more comprehensive and malleable snapshot of blood coagulation by providing, for the first time, at least one and preferably all of the following features: (a) the TFPI mediated inactivation of TF•VIIa and its product complexes, (b) the AT-III mediated inactivation of IIa, mIIa, factor VIIa, factor IXa, and factor Xa, (c) the initial activation of factor V and factor VIII by thrombin generated by factor Xa-membrane, (d) factor VIIIa dissociation/activity loss, (e) the binding competition, and kinetic activation steps which exist between tissue factor (TF) and factors VII and VIIa, and (f) the activation of factor VII by IIa, factor Xa, and factor IXa. By manipulating one or more of these parameters it is now possible to obtain predictive models of blood coagulation in silico

As discussed, there is almost universal recognition that the coagulation system is composed of a set of pro and anticoagulant systems that maintain the balance of blood fluidity. Defects in this balance can result in either thrombotic or bleeding tendencies e.g., hemorrhagic diseases (hemophilia A, hemophilia B, hemophilia C, para-hemophilia, hypoprothrombinemia) (1-6), thrombotic diseases, (antithrombin III (AT-III) deficiency, protein C deficiency, protein S deficiency and factor V^{Leiden}). See Mannucci, Pet al., 2001, N. Engl. J. Med. 344,1773-1779; Hoyer, 1994, N. Engl. J. Med. 330,38-47; Ozsoylu, et al., 1973, Acta. Haematol. 305-314; Poort et al., 1994, Thromb. Haemost. 72, 819-824; Bolton-Maggs, 1996,Baillieres Clin. Haematol. 9, 355-368; Chiu, et al., 1983, 72, 493-503; Beresford, 1988, Blood Rev. 2, 239-250; Reitsma, 1977, Thromb. Haemost. 78, 344-350; Griffin et al., 1987, Prog. Hematol. 15, 39-49; Bertina, et al., 1994, Nature 369, 64-67; Esmon, 1989, J. Biol. Chem. 264, 4743-4746; Mann, et al., 1995, Williams Hematology 5th edition 1206-1226, Lichtman, Coller and Kipps eds. McGraw-Hill, Inc.; Lawson, et al., 1993, Methods in Enzymology 222(Part A), 177-195; Esmon, 2000, Biochim. Biophys. Acta. 1477, 349-360; Broze, Jr., 1995, Thromb. Haemost. 74, 90-93; Davie, et al., 1991, Biochemistry 30,10363-10370. However, during thrombin generation by the tissue factor (TF)-initiated reaction, multiple transitory species are produced, many of which play multiple roles at different stages of the process. It is in the balance of this complex interplay, that the response to each injury or stimulus is most precisely determined.

Prior models of therapeutic antihemorrhagic and antithrombotic strategies have primarily relied upon intuitive approaches to judge how qualitative or quantitative alterations in a natural product or a therapeutic agent might behave in this complex reaction milieu. These estimates are usually made on the basis of oversimplifications, which ignore the dynamic interplay between coagulation factor reactions, and judgments are made based upon the presumption of isolated defective functions. In even the simplest cases, such as the single gene deficiencies (hemophilia A or B) in which a fundamental knowledge of the defect is available, less than satisfactory algorithms exist for determining the dosage required of the missing factor. See, Zoller, et al., 1999, Haematologica 84, 59-70; de Moerloose, et al., 1998, Semin. Thromb. Hemost. 24, 321-327.

In contra the invention provides a more complete understanding of the interplay between the pro and anticoagulant factors involved in hemostasis and should permit the kinetics of “composite” deficiencies to be better understood. The present invention extends prior understanding of thrombin generation reaction by including the stoichiometric inhibitor systems and by updating the mechanisms to the current level of knowledge. See, e.g., Jones, et al., 1994, J. Biol. Chem. 269, 23367-23373; Nesheim, 1984, J. Biol. Chem. 259, 1447-1453; Hockin, et al., 1999, Mann, 1999, Biochemistry. 38, 6918-6934; Mounts and Liebman, 1997, Int. J. Biol. Macromol. 20, 265-281; Beltrami, and Jesty, 1995, Proc. Natl. Acad. Sci. USA 92, 8744-8748; Gentry, et al., 1995, Biophys. J. 69, 356-61; Kessels, Het al., 1994, Comput. Biol. Med. 24, 277-288; Butenas, et al., 1999, Blood. 94, 2169-2178; Butenas, et al., 1997, 272, 21527-21533; van't Veer, et al., 1997, J. Biol. Chem. 272, 7983-7994; van't Veer and Mann, 1997, J. Biol. Chem. 272, 4367-4377; Rand, et al., 1996, Blood 88, 3432-3445; Brummel, et al, J. Biol. Chem. 274, 22862-22876; Baugh, et al., 1998, J. Biol. Chem. 273, 4378-4386; Jordan; et al., 1980, J. Biol. Chem. 255, 10081-10090; Chuang, et al., 20001, J. Biol. Chem. 276, 1496-14971; Lawson, et al., 993, J. Biol. Chem. 268, 767-770; Schoen and Lindhout, 1987, J. Biol. Chem. 262, 11268-11274; Lollar, et al., 1992, J. Biol. Chem. 267, 23652-23657; Fay, et al., 1992, J. Biol. Chem. 267, 13245-13250; Fay, P. J., et al., 1996, J. Biol. Chem. 271, 6027-6032; Butenas and Mann, 1996, Biochemistry. 1904-1910; Krishnaswamy et al., 1987, J. Biol. Chem. 262, 3291-3299; and Nesheim, et al., 1981, Methods Enzymol. 80 Pt C, 249-74. Practice of the invention provides further benefits, particularly replacing intuition with a rationally designed model for predicting blood coagulation. For example, increasing the concentration of a procoagulant factor such as Factor VII “should” lead to an increase in thrombin generation and decreased clotting time. But in both empirical studies and in laboratory experiments, the opposite is true (see, e.g., van't Veer, et al. 2000, Blood. 95, 1330-1335). Use of the invention provides the opportunity to integrate and quantify reaction details which, in turn, aids in the design of the more expensive empirical “wet” experiments.

Additionally, the preferred use of the invention allows a user to predict blood clotting in the face of diverse experimental conditions. The invention also provides the ability to look into reactions at a “microscopic” level, when the levels of substrates and products being investigated are below detection limits accessible through direct analysis. Such evaluations can provide estimations of the initiating events in a process and lead to the design of laboratory experiments, which are designed to explore unforeseen computational results.

Preferred practice of the invention can be performed by one or a combination of strategies. In one embodiment, simulations are initiated by “exposing” picoMolar (pM concentrations of TF to an electronic milieu consisting of factors II, IX, X, VII, VIIa, V, VIIII, and the anticoagulants tissue factor pathway inhibitor (TFPI), and antithrombin-III (AT-III) at concentrations found in normal plasma or associated with coagulation pathology. The reaction is followed to monitor thrombin generation and proceeds through phases that can be operationally defined as initiation, propagation, and termination. The generation of thrombin displays a non-linear dependence upon TF and TFPI and the combination of these inhibitors displays kinetic thresholds. For example, at sub-threshold TF, thrombin production/expression is suppressed by the combination of TFPI and AT-III. For concentrations above the TF threshold, the bolus of thrombin produced is quantitatively equivalent A comparison of the model with empirical laboratory data illustrates that most experimentally observable parameters are captured, and the pathology that results in enhanced or deficient thrombin generation is accurately described.

More specifically, preferred invention practice involves use of one or more computer programs (preferably adapted in a software format) that are designed to enable rapid transformation of chemical equilibrium expressions to the necessary time-dependent partial differential equations required for this model and their solution. In an especially preferred invention embodiment, that software package will sometimes be referred to herein as “Clotspeed.”

Typically, the Clotspeed package utilizes an Internet-based interface with a generally applicable fourth order Runge-Kutta solver that provides solutions to a family of time dependent differential equations. After the user inputs the chemical equations, initial concentrations and rate constants for all relevant species and steps, the software generates a series of time dependent concentration profiles for any/all reactants over any time frame of interest. A set of simulations can be developed representing titration of individual species or varying individual rate constants to represent qualitative or quantitative alterations in the reactant. The results of each simulation are stored in a relational database architecture utilizing SQL standards. For this work all computations were carried out on a Pentium III running LINUX (RedHat v. 7.1), however the software may be installed on any computer capable of running UNIX.

The validity of our implementation of the 4^{th} order Runge-Kutta solver can be tested in a variety of conventional ways. Preferably, validity is confirmed through use of the freely available BDF LSODE solver that utilizes 3^{rd} order polynomials (http://www.llnl.gov/casc/odeoak). The Livermore solver (LSODE) utilizes dynamic step sizing and a matrix of partial differential equations to approach the solution. Utilizing the Livermore algorithm, we were able to demonstrate exact correspondence to the solutions obtained with our Runge-Kutta solver. This correspondence was found between both simple chemical systems and numerous pre-publication versions of the coagulation model.

As discussed, the invention can be used to study and manipulate blood coagulation profiles. Preferred computer modeling programs used processes and rate constants that were generally representative of reaction paths and rates experimentally observed under the condition of saturating phospholipid concentrations. In the instances for which experimental data are not available for the processes modeled, rate constants and mechanisms were incorporated by analogy with similar processes within the coagulation cascade. The entire model, in tabular form, is presented as Table 1, below.

In Table 1, the notation −2> signifies a forward reaction dictated by rate constant “2” (Table 2). The notation <1-2> indicates an equilibrium expression with a forward rate constant of k₂ and a reverse rate constant of k₁. Binding between components is indicated by the=notation, i.e. A+B−<1-2>A=B.

TABLE 1
Chemical Expressions For The Coagulation Cascade

| Equation # | Chemical Expression |
|---|---|
| 1 | TF+VII<1-2>TF=VII |
| 2 | TF+VIIa<3-4>TF=VIIa |
| 3 | TF=VIIa+VII−5>TF=VIIa+VIIa |
| 4 | Xa+VII−6>Xa+VIIa |
| 5 | IIa+VII−7>IIa+VIIa |
| 6 | TF=VIIa+X<8-9>TF=VIIa=X−10>TF=VIIa=Xa |
| 7 | TF=VIIa+Xa<11-12>TF=VIIa=Xa |
| 8 | TF=VIIa+IX<13-14>TF=VIIa=IX−15>TF=VIIa+IXa |
| 9 | Xa+II−16>Xa+IIa |
| 10 | IIa+VIII−17>IIA+VIIIa |
| 11 | VIIIa+IXa<18-19>IXa=VIIIa |
| 12 | IXa=VIIIa+X<20-21>IXa=VIIIa=X−22>IXa=VIII+Xa |
| 13 | VIIIa<23-24>VIIIa₁·L+VIIIa₂ |
| 14 | IXa=VIIIa=X−25>VIIIa₁·L+VIIIa₂+X+IXa |
| 15 | IXa=VIIIa−25>VIIIa₁·L+VIIIa₂+IXa |
| 16 | IIa+V−26>IIa+Va |
| 17 | Xa+Va<27-28>Xa=Va |
| 18 | Xa=Va+II<29-30>Xa=Va=II−31>Xa=Va+mIIa |
| 19 | mIIa+Xa=Va−32>IIa+Xa=Va |
| 20 | Xa+TFPI<33-34>Xa=TFPI |
| 21 | TF=VIIa=Xa+TFPI<35-36>TF=VIIa=Xa=TFPI |
| 22 | TF=VIIa+Xa=TFPI−37>TF=VIIa=Xa=TFPI |
| 23 | Xa+ATIII−38>Xa=ATIII |
| 24 | mIIa+ATIII−39>mIIa=ATIII |
| 25 | IXa+ATIII−40>IXa=ATIII |
| 26 | IIa+ATIII−41 >IIa=ATIII |
| 27 | TF=VIIa+ATIII−42>TF=VIIa=ATIII |

Typically, literature values for rate constants are utilized as starting points. Additional fitting may be required either where values are evaluated under non-physiologic conditions or because they made use of extensively modified proteins. For example, the reported equilibrium constant for TF-factor VIIa-membrane can be estimated utilizing a truncated TF protein and evaluated by surface plasmon resonance utilizing chemical cross-linking to a substrate (see, e.g., O'Brien, et al., 1994, Biochemistry. 29, 14162-14169). In this case, the ratio of the reported reverse and forward rate constants (Kd) was maintained to fit published kinetic functional data (see, e.g., Krishnaswamy, 1992, J. Biol. Chem. 267, 23696-23706; Shobe, et al., 1999, J. Biol. Chem. 274, 24171-24175; Butenas, et al., 1994, Biochemistry. 33, 3449-3456).

The literature ranges and the rate constants used in the model are presented in Table 2 below.

TABLE 2
Listing Of Rate Constants UtiIized In The Model And
A Summary Of Literature References

| Rate | Model Value | Citation | Literature Range | Sec⁻¹ | M⁻¹sec⁻¹ |
|---|---|---|---|---|---|
| 1 | 3.1 × 10⁻³* | (A)(B) | 6.29 × 10⁻⁴-3.1x10⁻³ | • | |
| 2 | 3.2 × 10⁶* | (A)(B) | 3.14 × 10⁶-1.6 × 10⁵ | | • |
| 3 | 3.1 × 10⁻³* | (A)(C) | 8.76 × 10⁻⁴-1.6 × 10⁻³ | • | |
| 4 | 2.3 × 10⁷* | (A)(C) | 1.6 × 10⁵-1.9 × 10⁵ | | • |
| 5 | 4.4 × 10⁵ | (D) | | • | |
| 6 | 1.3 × 10⁷ | (D) | | | • |
| 7 | 2.3 × 10⁴ | (D) | | | • |
| 8 | 1.05* | Km in (C)(E) | 69 nM (PC/PS)-4.35 uM (PC) | • | |
| 9 | 2.5 × 10⁷* | Km in (C)(E) | 69 nM (PC/PS)-4.35 uM (PC) | | • |
| 10 | 6 | (F)(G) | 7.0-5.69 | • | |
| 11 | 19*(**) | none | | • | |
| 12 | 2.2 × 10⁷*(**) | (G)(H) | | | • |
| 13 | 2.4*^{(@)} | (I)(J) | 24 | • | |
| 14 | 1.0 × 10⁷*^{(@)} | (I)(J) | 1.0 × 10⁸ | | • |
| 15 | 1.8*^{(@)} | (I)(J) | 0.3 | • | |
| 16 | 7.5 × 10³ | (in prep) | | | • |
| 17 | 2.0 × 10⁷ | (E) | | | • |
| 18 | 5.0 × 10⁻³ | (I) | | • | |
| 19 | 1.0 × 10⁷ | (I) | | | • |
| 20 | 1.0 × 10⁻³ | (I) | | • | |
| 21 | 1.0 × 10⁸ | (I) | | | • |
| 22 | 8.2 | (I) | | • | |
| 23 | 2.2 × 10⁴* | (K)(L) | Based on (K) and Kd (L) | | • |
| 24 | 6.0 × 10⁻³ | (K) | | • | |
| 25 | 1.0 × 10⁻³ | (M) | | • | |
| 26 | 2.0 × 10⁷ | (I) | | | • |
| 27 | 0.2 | (I) | | • | |
| 28 | 4.0 × 10⁸ | (I) | | | • |
| 29 | 103 | (I) | | • | |
| 30 | 1.0 × 10⁸ | (I) | | | • |
| 31 | 63.5 | (I) | | • | |
| 32 | 1.5 × 10⁷ | (I) | | | • |
| 33 | 3.6 × 10⁻⁴ | (E) | | • | |
| 34 | 9.0 × 10⁵* | (E) | 1.35 × 10⁶ | | • |
| 35 | 1.1 × 10⁻⁴* | (E) | 1.1 × 10⁻³ | • | |
| 36 | 3.2 × 10⁸* | (E) | 2.72 × 10⁸ | | • |
| 37 | 5.0 × 10⁷* | (E) | 7.34 × 10⁶ | | • |
| 38 | 1.5 × 10³ | (N) | | | • |
| 39 | 7.1 × 10³ | (O) | | | • |
| 40 | 4.9 × 10² | (N) | | | • |
| 41 | 7.1 × 10³ | (O) | | | • |
| 42 | 2.3 × 10² | (P) | | | • |

*Indicates that the rate constant used in the model was derived through fitting of computational results to empirical data, these values were usually seeded with literature values when available or generated based upon analogy to other known similar processes.
(**)References G and F provide evidence that factor Xa or similar proteolytic fragments of factor Xa will bind the TF=VIIa complex with affinity similar factor X; the rate constants illustrated are derived based on this assumption and are not contained in the reference.
(^{@})These values were based upon a knowledge of the rates for factor X activation by TF=VIIa as referenced, and the relative ratio between the rate of activation of X vs. IX in a TF dependent system (Ref J).
References are listed in alphabetical order:
(A) O'Brien, et al, 1994, Biochemistry. 33, 14162-14169;
(B) Krishnaswamy, 1992, J. Biol Chem. 267, No. 33, 23696-23706;
(C) Shobe, et al, 1999, J. Biol Chem. 274, No. 34, 24171-24175;
(D) Butenas and Mann, 1996, Biochemistry 35, 1904-1910;
(E) Baugh, et al., 1998, J. BioL Chem. 273, No. 8, 4378-4386;
(F) Braugh, et al., 2000, J. BioL Chem. 275, No. 37. 28826-28833;
(G) Baugh and Krishnaswamy, 1996, J. Biol Chem. 271, No. 27. 16126-16134;
(H) Bergum, et al., 2001, J. Biol. Chem. 276, No. 13, 10063-10071;
(I) Jones and Mann, 1994, J. Biol Chem, 269, No.37, 23367-23373;
(J) Bom, et al., 1991, Thromb Haemo. 66, No. 33., 283-291;
(K) Lollar, 1992, J. Biol Chem. 267, No. 33, 23652-23657;
(L) Fay and Smudzin, 1992, J. Biol Chem. 267, No. 19, 13245-13250;
(M) Fay, et al., 1996, J. Biol. Chem. 271, No. 11, 6027-6032;
(N) Chuang, et al., 2002, J. Biol. Chem. 276, No. 18, 14961-71;
(O) Schoen and Lindhout, 1987, J. Biol. Chem. 262, No. 23, 11268-74;
(P) Lawson, et al., 1993, J. Biol. Chem. 268, No. 2, 767-70.

TABLE 3
Mean Plasma Concentrations of Pro- and Anticoagulants

| Species | Initial Concentration (Model) |
|---|---|
| TF | Varied |
| VII | 1.0 × 10⁻⁸ |
| TF=VII | 0.0 |
| VIIa | 1 × 10⁻¹⁰ |
| TF=VIIa | 0.0 |
| Xa | 0.0 |
| IIa | 0.0 |
| X | 1.6 × 10⁻⁷ |
| TF=VIIa=X | 0.0 |
| TF=VIIa=Xa | 0.0 |
| IX | 9 × 10⁻⁸ |
| TF=VIIa=IX | 0.0 |
| IXa | 0.0 |
| II | 1.4 × 10⁻⁶ |
| VIII | 0.7 × 10⁻⁹ |
| VIIIa | 0.0 |
| IXa−VIIIa | 0.0 |
| IXa=VIIIa=X | 0.0 |
| VIIIa₁−L | 0.0 |
| VIIIa2 | 0.0 |
| V | 2.0 × 10⁻⁸ |
| Va | 0.0 |
| Xa=Va | 0.0 |
| Xa=Va=II | 0.0 |
| mIIa | 0.0 |
| TFPI | 2.5 × 10⁻⁹ |
| Xa=TFPI | 0.0 |
| TF=VIIa=Xa=TFPI | 0.0 |
| ATIII | 3.4 × 10⁻⁶ |
| Xa=ATIII | 0.0 |
| mIIa=ATIII | 0.0 |
| IXa=ATIII | 0.0 |
| IIa=ATIII | 0.0 |
| TF=VIIa=ATIII | 0.0 |

To best understand the invention, it is convenient to define the coagulation reaction leading to thrombin generation in three phases: initiation, propagation, and termination. This “working simplification” provides a convenient vehicle for accomplishing this goal.

A. Initiation: The extrinsic factor Xase (factor VIIa-TF-membrane) forms through the assembly of membrane associated TF and factor VII/VIIa and was modeled as an equilibrium process (Table 1, # 1-2). Rate constants for this process were estimated by analogy with the assembly processes for the intrinsic factor Xase (factor Xa-factor VIIIa-membrane) and prothrombinase (factor Xa-factor Va-membrane) (Table 2). The extrinsic factor Xase functions by the activation of factor X (Table 1, # 6-7), factor IX (Table 1, # 8) and in a self-propagating loop the activation of factor VII (Table 1, # 3). Factor VII activation also occurs by thrombin and factor Xa (Butenas and Mann, 1996, Biochemistry. 1904-1910) and is included (Table 1, # 4-5) in this version of the model.

Empirical analyses of early events during the TF initiated generation of thrombin, by Butenas, et al., 1994, supra, convincingly demonstrated that early factor V activation occurs exclusively by thrombin. Further, anisotropy data for factor Xa binding show that factor V at biologically relevant concentrations (20 nM) does not bind factor Xa and thus cannot participate in prothrombinase formation prior to its proteolytic activation. An evaluation of the activation of prothrombin by factor Xa-phospholipid in the absence of factor Va at 37° at enzyme concentrations relevant to the initiation phase of the reaction gave K_{M} 0.3±0.05 nM and k_{cat} 2.3×10⁻³ sec⁻¹. Accordingly, in the model the initial factor Va is produced by thrombin activation (Table 1, #16) with the latter activated by factor Xa-PCPS (Table 1, # 9) using the appropriate constants (Table 2, # 16). The activation of factor VII is as previously described while a chemical mechanism for factor VIIIa activity loss by spontaneous A₂ domain dissociation (Lollar, et al., 1992, J. Biol. Chem. 267, 23652-23657; Fay and Smudzin, 1992, J. Biol. Chem. 267, 13245-13250; Fay, et al., 1996, (J. Biol. Chem. 271, 6027-6032) (Table 1, #13,15) is incorporated. The rate constants for these dissociation processes and their governing binding constants at physiological pH are utilized (Table 2, #23-25) (Fay, et al., 1996, J. Biol. Chem. 271, 6027-6032).

B. Propagation: The production of factor Xa by both the intrinsic and extrinsic factor Xases is as previously described as is the formation and activity of the prothrombinase complex (14) (Table 1, #6-8, 17). The thrombin self-propagation by activation of components of the complexes catalyzes its formation is implemented (Table 1, # 5, 10, 16). The thrombin precursor, meizothrombin (mIIa), is less efficient at these processes and was therefore not included as a separate entity (Doyle and Mann, 1990, J. Biol. Chem. 265, 10693-10701).

C. Termination: The stoichiometric inhibitors AT-III and TFPI are included, enabling the analyses of their isolated and combined effects on thrombin generation. The contribution of AT-III is straightforward using existing literature for the second-order rate constants for AT-III inhibition of IIa, factor Xa, factor VIIa, and factor IXa (Table 1, # 23-27). TFPI inhibition proved challenging. This inhibitor has been the subject of numerous investigations. The most satisfactory explanation for TFPI behavior is provided by Baugh, et al., 1998, J. Biol. Chem. 273, 4378-4386, and the invention accommodates their scheme (Table 1, # 20-22) and rate constants (Table 2, # 33-37).

TFPI acts through two pathways, one of which involves the inhibition of the enzyme product complex TF•VIIa•Xa (Table 1, # 21). The other pathway involves a three step mechanism: 1) inhibition of the product factor Xa by TFPI (Table 1, # 20); 2) binding of the Xa•TFPI complex to the TF•VIIa complex through the substrate interaction domain of factor Xa, (Table 1, #22; and 3) the inhibition of the bound TF•factor VIIa through a first order “rearrangement” wherein the Kunitz domain of TFPI interacts with the factor VIIa active site (Baugh et al., 1998, supra), producing a product indistinguishable from that of the second order addition to the enzyme-product complex.

Implementation of the second order, enzyme-product-TFPI pathway (Table 1, #21) is straightforward using the published rate constants. The alternative mechanism creates problems due to the unimolecular inhibition process as the final step. Initial attempts at modeling this system led to computational instability that was relieved by condensing the final two steps of this mechanism into a single second order collisional process dictated by limiting the forward or reverse rates of the two steps. This eliminates the problems associated with interpreting a collisional rate constant in the context of a first order process. Confirmation of the validity of this approach was obtained through construction of a model involving only the extrinsic factor Xase components and TFPI. This TFPI model generated data comparable to empirical TFPI inhibition rate data.

Unless otherwise indicated, all simulations are performed utilizing the mean plasma concentrations for all proteins (Table 3): prothrombin (1.4 μM), factor X (160 nM), factor IX (90 nM), factor V (20 nM), factor VII (10 nM), factor VIIa (100 pM), factor VIII (700 pM, TFPI (2.5 nM, and AT-III (3.4 μM). TF concentrations are varied between 1-25 pM to simulate estimates of a physiologically relevant challenge (Shobe, et al., 1999, J. Biol. Chem. 274, 24171-24175). Total thrombin (IIa and mIIa) with units corresponding to thrombin-seconds is obtained by integrating the thrombin concentration over an experimental time interval. This value represents the quantitative exposure of the experimental system to thrombin activity. The model was tested by simulation of experimental conditions that have shown unique thrombin profiles (Butenas, et al., 1999, Blood. 94, 2169-2178; van't Veer and Mann, 1997, J. Biol. Chem. 272, 4367-4377). Comparisons between simulations and experimental data can be used in assessing the fidelity of the model to the empirical system.

The present invention is readily refined as follows. Analysis of the model TFPI mechanism included tests using the isolated inhibitory loop in simulations involving only, TF, factor VIIa, TFPI and factor X, in which the amount of factor Xa formed over time can be determined. In a typical simulation, the TF concentration is varied from about 1-1024 pM while the factor VIIa (2 nM), TFPI (2.5 nM), and factor X (170 nM) are held constant. The factor Xa profiles re contrasted with published data and the model parameters adjusted until correspondence is obtained. Further simulations in which the initial conditions included preformed TFPI=factor Xa complexes can be conducted to verify the response. The effect of TFPI can be explored in terms of thrombin response, lag phase, and maximal rate of thrombin generation at multiple TF concentrations. Independent titrations of TFPI (0-150 nM), TF (0.01-1000 pM) and factor VIIa (0.1-20 nM), under the set of otherwise normal concentrations (Table 3) can be conducted as well as simulations examining the thrombin generation profile for conditions mimicking severe hemophilia A (zero factor VIII) in the presence or absence of TFPI. The results of each set of simulations can be compared to experimental data (Baugh, et al., 1998, 273, 4378-4386).

Validation of the model is conducted as follows. The aggregate effect of TFPI and AT-III on the procoagulant model is assessed by including both inhibitors in the procoagulant model. Initial simulations were conducted to evaluate the thrombin response profile, and the integrated thrombin levels generated after stimulus with TF over a range spanning 0.01-1000 pM. Subsequent analyses include examination of the thrombin response in hemophilia A at various TF stimuli (1, 5 and 25 pM TF) at various factor VIII concentrations (100%, 10% and 1% factor VIII). Further analyses of the hemophilia A conditions are conducted quantifying the thrombin response to factor VIIa titration in severe hemophilia (<1% factor VIII). Simultaneous variations in the level of AT-III within the clinically normal range (50% or 150%) and in the level of II (150% or 50%) are analyzed at 5 pM and 25 pM TF and the results contrasted with published reports (see, Examples below).

In one preferred embodiment, the invention provides a method of modeling the effect of an agent on a molecular pathway, such as blood coagulation. FIG. 11 is a block diagram representing the logic sequence for a software program that predicts the speed and efficacy of an agent in clotting blood, according to one aspect of the invention. Beginning at step S1, a user logs in with a user name and password. Program flow then continues at step S2, where the user may select existing equations from a prior run of the program. If the user selects the existing equations, program flow continues at step S3, where the program creates new IDs for the species and rate constants. Program flow then continues at step S4.

If the user does not select the existing equations, program flow continues at step S4, where the user inserts. new equations into the database. Program flow then continues at step S5, where a subroutine, called the “solver,” breaks down the equations to individual species and rate constants. Program flow then continues at step 56, where the user selects new or old species. If the user selects the new or old species, program flow continues at step S7, where the new or old species is inserted into the database. Program flow then continues at step S8. If the user does not select the new or old species, program flow bypasses step S7, and continues at step S8.

At step S8 a user selects new or old rate constants. If the user selects new or old rate constants, program flow continues at step S9, where new rate constants are inserted into the database. Program flow then continues at step S10. If the user does not select new or old rate constants, program flow bypasses step S9 and continues at step S10.

At step S10 all data used for the calculations is stored in a text file. At step S11, the solver parses the text file created in step S10, creates the corresponding equations, and solves them. Program flow then continues at step S12, where the results of the calculations are saved to the database.

Program flow then continues at step S13, where the user selects whether to display the data on the monitor as graphical data. If the user does not wish to display the data on the monitor, program flow continues at step S14, where the data is output to an Excel® formated file. If the user wishes to display the data on the monitor as a graph, program flow continues at step S15, where the data is displayed. After display or output in step S15 or S14, respectively, program flow terminates at step S16.

In one practical embodiment, a program using the logical steps of the flowchart of FIG. 11 was written where a user could enter data into the program and calculate blood coagulation speed and efficacy. The program was written in C++. However, in order to create an easy method for entering data and understanding the results, an interface was developed for the Web using a combination of PHP, standard html, and Portable Networks Graphic (PNG) for producing graphs. A database, mySQL, was also used to facilitate the handling and organization of the large volume of data produced. The program was run on a personal computer (PC) running a Linux operating system with Apache HTTP server software, mySQL and the PNG libraries.

One of the programs or scripts was called index.html, and it was used for the Apache server username/password recognition operation.

One program or script was called index2.html and it was used for the database username/password recognition operation.

One program or script was called menu.php program, and it was used to create a menu in the left frame of the web page. The menu contained the following operations:
Back to Login (using the program or script index2.html)
Start model (using the program or script view.php)
Delete Section (old data, using the programs or scripts delete.php→delete2.php)
Graph Old Data (using the programs or scripts view2.php→graphing.php)
Text of Old Data (using the programs or scripts view3.php→text.php→text2.php)
For Excel® Data (exports data in Excel® format, using the programs or scripts ToExcel.php→ToExcel2.php→ToExcel3.php→ToExcel4.php)
   The programs mentioned above will be discussed more fully in connection with the modeling function, below.

One program or script was called view.php, and it provided a user with the ability to select to use another user's input files, such as his/her equations, initial species concentrations and rate constants.

Another program or script was called eq.php, and it provided the user with the ability to select old equations, either his/her own or another's, depending on what was entered with program or script view.php. The user could also select to create new equations if desired.

One program or script was called eq2.php. The program eq2.php displayed in a text box either equations from a prior run (as selected in eq.php) or an empty text box for the insertion of new equations. The user was then prompted to select a set of prior species concentrations from his/her own prior runs or another's initial species concentrations, or to enter new species concentrations. It will be appreciated that the user indicated the choice of whose data would be accessible from the program or script view.php.

One program or script was called rc.php, and it was first used to insert equations into the database if the equations were new and/or modified by the program or script eq2.php. In this instance, if the user was not the owner of the equations, the equations were copied from the original owner's database to the user's database. This program or script then passed the equations to another program, the solver.

The solver, written in C++, parsed the equations into individual species and rate constants. The parsed equations were then grouped by species and the rate constants present The solver program returned these grouped species and rate constants back to the program or script rc.php.

The program or script rc.php then displayed to the user the equations involved in producing a given species, and either displayed the initial concentration for that species (as chosen in eq2.php) or provided an empty field for the user to enter new concentrations. Once the user had entered all of the initial species concentrations, the user was prompted to select a set of prior rate constants from his/her own prior runs, another's rate constants, or to enter new rate constants. It will be recalled from the prior discussion that the user indicated the choice of whose data would be accessible from the program or script view.php.

Another program or script was called rc1.php, and it inserted the initial species concentrations into the database if the values were new and/or modified. The equation using the rate constant was presented to the user with either the initial value determined from the user's selection in rc.php, or an empty field for the user to enter a new rate constant

Another program or script was called rc2.php, and it inserted rate constants into the database if the rate constant values were new and/or modified. The script or program prompted the user for input on the duration of the experiment and whether, in this experiment, a titration with either the initial concentrations of species or the rate constants was desired. If the user chose to run a titration experiment, he/she was provided with the option of selecting the following values: the individual species or rate constant to modify, high/low (150%, 100%, 50% of value entered previously) or user defined. In the user defined category, as many as six (6) values could be entered.

The user was also prompted to select up to twelve (12) species to follow, either graphically or textually. Users could also elect to sum final species concentrations.

The program also permitted an advanced user to modify the differential equation solver step size.

Another program or script was called exe.php, and it executed the solver program the number of times specified by the data entered with the program or script rc2.php.

Another program or script was called temp.php, and it stored data generated from each execution of the solver in the database. The logfiles for each run were made accessible to the user to facilitate troubleshooting. The user was prompted for a title for this experiment, in order to differentiate it from previous ones. It will be appreciated that this run became the value that was prompted to the user in view.php to select old data. The temp.php program or script also permitted the data to be viewed in either graphical or textual format. If the user chose a graphical display, then the program temp.php offered choices for how the data could be viewed (i.e. individual graphs of each species monitored or combined graphs where multiple species are monitored). Another option permitted the user to plot his/her data in conjunction with another user's data

Another program or script was called run2.php, which provided the user with an option to view plotted graphs made in the program or script temp.php. It also permitted the user to create new graphs with selected data from either the user's earlier experiments or another's experiments. Another program or script, called graph.php, was responsive to run2.php, and actually drew the PNG graph in a new window.

Another program or script was called run3.php, and it was used to combine new data with data from prior runs or with another's data. The aforementioned program, graph.php, responded to the program run3.php, and actually drew the PNG graph in a new window.

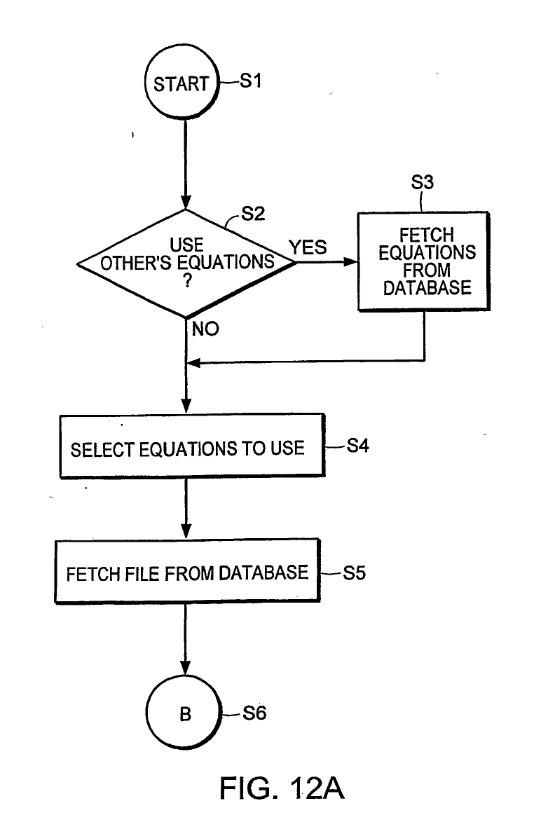

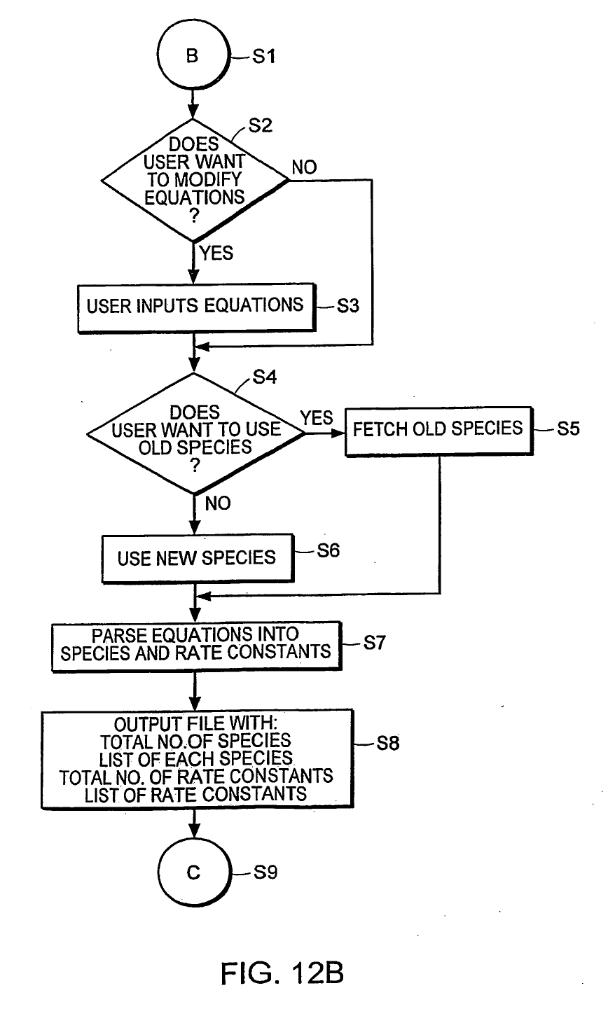

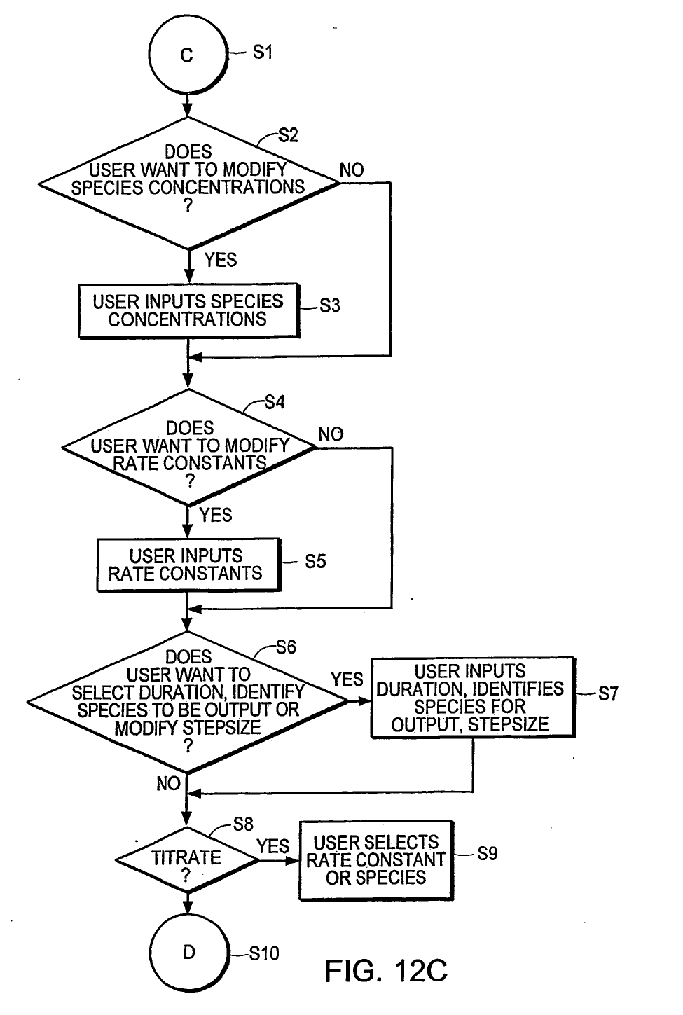

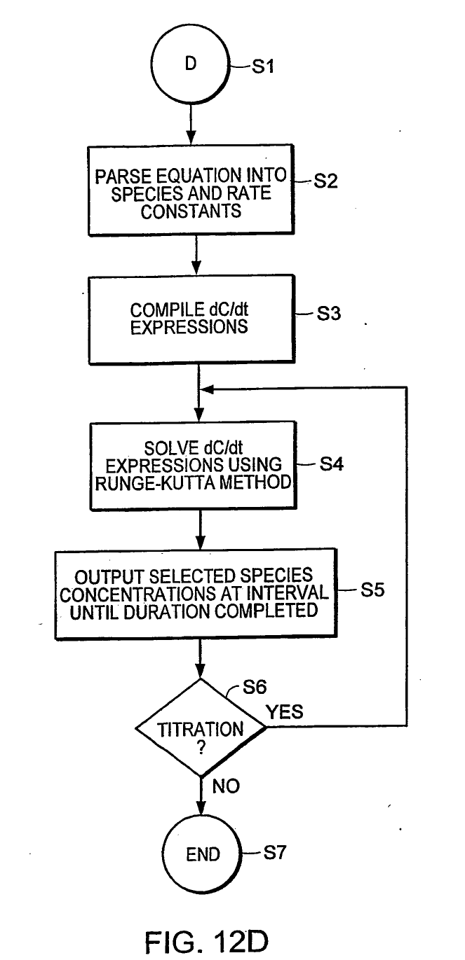

The solver program of FIG. 11, detailed more fully in the flowcharts of FIGS. 12A to 12D, was the result of a mathematical model that was created to aid in the understanding of the blood coagulation system by modeling the kinetics of the enzyme linked systems. The solver was limited to second order reactions, since its primary design was for use in biochemistry models. As the collective understanding of the coagulation cascade matured, the model system evolved into ever greater complexity, requiring the simultaneous solution of systems of differential equations describing independent rate processes for over twenty (20) components. The ever increasing complexity of the system led to a bookkeeping problem, however. Given a single component, e.g., thrombin, which participates in five (5) separate processes related to its formation or consumption, alteration of any one of these processes through inclusion of alternative substrates or fates required changes in a number of independent dC/dt expressions. Thus, working by hand to re-write dC/dt expressions for numerical solutions became time consuming, and led to repeated periods of testing, wherein the simulations were run and rerun to insure that all equilibrium expressions were satisfactory and all dC/dt expressions included the necessary components. To overcome the inevitable human errors introduced into this process, a software package was developed that would take mathematical equations, develop the dC/dt expressions, and solve the system of equations at defined time points. With the continuous increase in CPU speed, and the evolution of the World Wide Web as a universal programming interface, a solver system was created that was universally applicable to all kinetic systems, and was then tailored to the chemistry of blood.

The equations used in the software program of FIGS. 12A to 12D were written in the following format:
A+B<1-2>AB (1)
AB−3>A+C (2)

In the software, these equations were used to derive the fundamental equilibrium dC/dt expressions using the rules described here within:

$$\begin{matrix}\frac{ⅆ A}{ⅆ t} = - [ A ] [ B ] {k}_{2} + [ A B ] ( {k}_{1} + {k}_{3} ) & ( 3 ) \\ \frac{ⅆ B}{ⅆ t} = - [ A ] [ B ] {k}_{2} + [ A B ] {k}_{1} & ( 4 ) \\ \frac{ⅆ A B}{ⅆ t} = [ A ] [ B ] {k}_{2} - [ A B ] ( {k}_{1} + {k}_{3} ) & ( 5 ) \\ \frac{ⅆ C}{ⅆ t} = [ A B ] {k}_{3} & ( 6 )\end{matrix}$$

The software package utilized the format of equations (1-2) to generate expressions (3-6), thus eliminating human error often introduced when preparing these expressions by hand. The software prompted the user for initial concentrations and rate constant values. These values were then used to model the linked system and generate anticipated time dependent concentration values for each species at user specified intervals. The software performed rapidly, and, in one practical embodiment generated time dependent concentration values for a system utilizing twenty-five (25) species tracked for five (5) minutes at one (1) second intervals in approximately fifteen (15) seconds.

Most chemical kinetic problems posed a significant difficulty for numerical analysis because the functions that described the decay or appearance of a specific species contained variable species concentration on each side f the expression, as well as multiple species on the right hand side (RHS). Further mathematical difficulties resulted from the necessity of including three separate dC/dt expressions to account for a single species. For example, species A in expressions 3-5:

$$\begin{matrix}\frac{ⅆ A}{ⅆ t} = - [ A ] [ B ] {k}_{2} + [ A B ] ( {k}_{1} + {k}_{3} ) & ( 3 ) \\ \frac{ⅆ B}{ⅆ t} = - [ A ] [ B ] {k}_{2} + [ A B ] {k}_{1} & ( 4 ) \\ \frac{ⅆ A B}{ⅆ t} = [ A ] [ B ] {k}_{2} - [ A B ] ( {k}_{1} + {k}_{3} ) & ( 5 )\end{matrix}$$

In these expressions, [A] appeared on both the left and right side. It will be appreciated that dA/dt was explicitly written as d[A]/dt, while the RHS contained the unique species [A], [B], and [AB]. It will also be appreciated that each expression defined a separate component uniquely affecting dA/dt. By contrast, the first order rate problem encountered in radioactive decay calculations was as follows:

$$\begin{matrix}{}_{86}^{222} Rn - 1 > {}_{86}^{218} Po & ( 7 ) \\ \frac{ⅆ {}_{86}^{222} Rn}{ⅆ t} = - [ {}_{86}^{222} Rn ] {k}_{1} & ( 8 ) \\ \frac{ⅆ {}_{86}^{218} Po}{ⅆ t} = [ {}_{86}^{222} Rn ] {k}_{1} & ( 9 )\end{matrix}$$

In the above example, the expression for d²²²Rn/dt was explicitly solvable and could have been written as:

$$Ln ( \frac{No}{N} ) = {k}_{1} t \qquad ( 10 )$$

where equation (10) was the result of explicit differentiation of the rate equation (7), yielding a simple exponential problem. Due to the nonlinear character of expressions (3-6), no such explicit solution was available. It will be appreciated that an infinite Taylor series expansion could have been utilized to indicate solutions to the differential expressions (3-6), and that computational algorithms based upon the Taylor series could have been utilized to describe the next step of a function y through its course:

$${y}_{n + 1} = {y}_{n} + \frac{ⅆ y}{ⅆ x} {\vert}_{n} \frac{Δ x}{1 !} + \frac{{ⅆ}^{2}}{ⅆ {x}^{2}} {\vert}_{n} \frac{Δ {x}^{2}}{2 !} + \frac{{ⅆ}^{3}}{ⅆ {x}^{3}} {\vert}_{n} \frac{Δ {x}^{3}}{3 !} + … + … \qquad ( 11 )$$

Thus, if the instantaneous derivatives of a function y were known at any point, the value at points on either side could be evaluated through successive approximations using higher order terms. However, explicit solutions to ordinary differential equations were sparse for nonlinear systems. If an approximation/adaptation of the Taylor Series (11) was used, known as the explicit fourth order Runge-Kutta method, an approximate solution to the generalized initial value problem (12-13) was:

$$\begin{matrix}\frac{{ⅆ}^{->} y}{ⅆ t} = {f}^{->} \text{(} t , {y}^{->} \text{)} \\ where & ( 12 ) \\ {y}^{->} ( t = {t}_{0} ) = {{y}^{->}}_{0} & ( 13 )\end{matrix}$$

Expressions (3-6) fell into the generalized description of initial value problems for which expressions (12-13) described, and approximate solutions could be found, using the Runge-Kutta algorithm. Known ordinary differential equations (ODE) analysis procedures for such systems included the Taylor method of order 4, the double precision 4th order Runge-Kutta solver, FORTRAN 77 (implemented in the preferred embodiment), as well as Hindmarsh's LSODE solver. The classical or 4th order Runge-Kutta algorithm utilized the following approach to accelerate its rate of convergence:

$$\begin{matrix}{y}_{n + 1} - {y}_{n} = \frac{Δ t}{6} ( {k}_{1} + 2 {k}_{2} + 2 {k}_{3} + {k}_{4} ) \\ where & ( 14 ) \\ {k}_{1} = \frac{ⅆ y}{ⅆ t} {\vert}_{{t}_{0 ,} {y}_{0}} & ( 15 ) \\ {k}_{2} = \frac{ⅆ y}{ⅆ t} {\vert}_{{t}_{0} + \frac{Δ t}{2} , {y}_{0} + \frac{Δ {tk}_{1}}{2}} & ( 16 ) \\ {k}_{3} = \frac{ⅆ y}{ⅆ t} {\vert}_{{t}_{0} + \frac{Δ t}{2} , {y}_{0} + \frac{Δ {tk}_{2}}{2}} & ( 17 ) \\ {k}_{4} = \frac{ⅆ y}{ⅆ t} {\vert}_{{t}_{0} + Δ t , {y}_{0} + Δ {tk}_{3}} & ( 18 )\end{matrix}$$

Hindmarsh's LSODE was based: on Backward Differentiation Formula (BDF) methods, mostly using 3rd order polynomial, but took control of the step size, and thus resulted in a more efficient computation. A reasonably accurate solution for Runge-Kutta could have been obtained for many fictions, provided a sufficiently small step size in time, dt, was utilized.

One important, but necessary, condition for the evaluation of the set of differential expressions (3-6)-was that the initial values concentration, in this case, be known and that the rate constants were defined appropriately. The software utilized equations (1-2) to determine the number of unique reactants, products, and rate constants. These unique species were then presented in a web interface with a text box requiring user entry of the values for the initial concentration of each species and each rate constant It will be appreciated that, in the case of the coagulation model, most values for the initial concentration of each species were zero.

Since the Runge-Kutta method utilized defined step intervals (in kinetics, this interval was time) to approach an experimental solution, the success of this method was largely dependent upon a careful choice of stepsize. The stepsize value needed to be optimized appropriately in order to maximize computational utility and experimental value. The use of large (greater than one (1) second) intervals was found to result in computational instability, resulting in the return of zero or NaN (not a number, i.e., infinite) values for the concentration of a few or most species of interest. Routine use of 0.01 second intervals resulted in computational stability for the coagulation simulations, and yielded values identical to similar type solvers running on diverse computer systems with diverse compilers, such as R10K Silicon Graphics Inc. (SGI) Octanes, and PII, PIII, and PPC machines running the Linux operating system. This approach required a large number of computational cycles, as the number of iterative calculations required in the solution of any equation system had to be non-linearly proportional to the order of the calculation. In one practical embodiment, the software was run with a start of 0.01 seconds, with the stepsize then decreased by a factor of ten. The results were then compared. If both were identical, it was not necessary to reduce the stepsize further. If computational instability was apparent, the stepsize was decreased again, and the iteration was repeated.

It will be appreciated that there was one further consideration regarding stepsize and data representation. The program requested a duration interval from the user, which did not affect the computational algorithm. The software calculated the species concentrations every tenth (0.1) second. Thus, a twenty (20) minute simulation generated 12,000 data points for each species. In the case of twenty (20) species, this resulted in 240,000 data points. Therefore, by default, the software generated data points at one (1) second intervals. It will be further appreciated that no round off error was introduced, as this method simply defined the data points to be output

The software program, in accord with the present invention, defined a rigorous system of inputting chemical equations in a manner that allowed the computer to model the relationships among reactants, and products with their rate constants. The program was designed such that reversible equilibrium expressions could be input as a single line, i.e., there was no need independently to define the forward and reverse reactions using separate lines. The limits imposed upon the scripting language were:

1. Species names were limited to 20 characters in length and could contain any character string with the exception of the +,>, <, and − characters without spaces. The equation parser was case sensitive, i.e., a distinction was made between capital letters and lowercase letters. The characters >, <, and − were reserved to denote the direction of reaction progress and rate constant identity, as discussed more fully hereinbelow.

2. The total number of unique species in any system was limited to 1000.

3. The total number of rate constants was limited to 1000.

4. The description of rate constants had to be numeric integers of value less than 100. This was for the rate constant description, and not its value.

5. The maximum number of species that could be included in one side, e.g., on the RHS, of a single expression, was 5.

6. There was a limit of 70 characters per line in total. Accordingly, economy in notation was important for the system. It is to be recalled that the equation compile was case sensitive, so that IIa and iia were considered distinct species.

The following are examples of irreversible and reversible systems of equations that assisted in a complete understanding of the scripting notation used in the software program. In general, the following text objects were used to describe forward reactions, reverse reactions, and equilibrium expressions.

−1> Reaction proceeded from left to right, as written, according to the rate constant 1.

<2− Reaction proceeded from right to left, as written, according to the rate constant 2.

<1-2> The reaction was an equilibrium expression proceeding left, according to the rate constant 1, and right, according to the rate-constant 2.

The use of these text objects is shown below:

Irreversible reaction: A+B−1>D

Irreversible reaction: D<1−A+B

Equilibrium Expression A+B<2-1>C−3>D

When these reactions were entered, the computer requested initial values for the species A, B, C, D, and then for the rate constants 1, 2, and 3.

Turning now to FIG. 12A, the logic sequence for a software program for the solver begins at step S1, and proceeds to a decision step, step S2, where the program interrogates the user as to whether to use another user's equations. If the user wishes to use another user's equations, program flow continues at step S3, where the program fetches a file containing the equations from the database, where they were saved from a previous run. If the user does not wish to use another's equations, program flow continues at step S4, where the user selects from the equations to be used for a run. At step S5, the program fetches the file from the database with the selected equations. Program flow then continues at step S6, or “B”.

Referring now to FIG. 12B, program flow continues at step S1 or “B”, and then proceeds to step S2, where the program interrogates the user as to whether the user wishes to modify the selected equations. If the user wishes to modify the selected equations, program flow continues at step S3, where the user inputs the modified equations. Program flow then continues at step S4. If the user does not wish to modify the equations at step S2, program flow continues at step S4. At step S4, the program interrogates the user as to whether the user wishes to user old species. If the user wishes to use old species, program flow continues at step S5, where the program fetches the old species from the database. Program flow then continues after step S6. If the user wishes to use new species from step S4, program flow continues at step S6, where the new species are used. At step S7, the solver parses the equations into species and rate constants. Program flow then continues at step S8, where the program creates an output file with the total number of species, a list of all species, with one species on each line, the total number of rate constants, and a list of rate constants, with one rate constant on each line. Program flow then continues at step S9, or “C”.

Turning to FIG. 12C, program flow begins at step S1 or “C”, and then proceeds to step S2, where the program interrogates the user as to whether the user wants to modify the species concentrations. If the user wishes to modify the species concentrations, program flow continues at step S3. Otherwise, program flow continues at step S4. At step S4, the program interrogates the user as to whether the user wishes to modify the rate constants. If the user wishes to modify the rate constants, program flow continues at step S5, where the user inputs the rate constants. Otherwise, program flow continues at step S6. At step S6, the program interrogates the user as to whether the user wishes to select the duration, to identify the species to be output or to modify the stepsize. If the user desires to select the duration, to identify the species to be, output, or to modify the stepsize, program flow continues at step 7, where the user inputs the duration, the species to be identified on output, and the modified stepsize. Program flow then continues at step S8. If the user chooses not to make any selections at step S6, program flow continues at step S8, where the program interrogates the user as to whether a titration is desired. If the user desires a titration, program flow continues at step S9, where the user selects the rate constants or species for the titration. The user also selects modify, high/low (150%, 100%, 50% of value entered previously) or user defined for the titration. Program flow then continues at step S10. or “D”. If the user does not select titration at step S8, program flow continues at step S10.

Referring to FIG. 12D, program flow begins at step S1 or “D”, and proceeds to step S2. At step S2, the solver parses the equations into the species and rate constants. Program flow then continues at step S3, where the program compiles the dC/dt expressions. At step S4, the program solves the dC/dt expressions using the Runge-Kutta method. The program then outputs, at step S5, a file with the selected species concentrations at the selected interval until the selected duration is completed. Program flow then continues at step S6, where it is determined if the user selected a titration. If the user selected a titration, program flow returns to step S4, where steps S4 and S5 are repeated until the titration is completed. If no titration was selected, program flow ends at step S7.

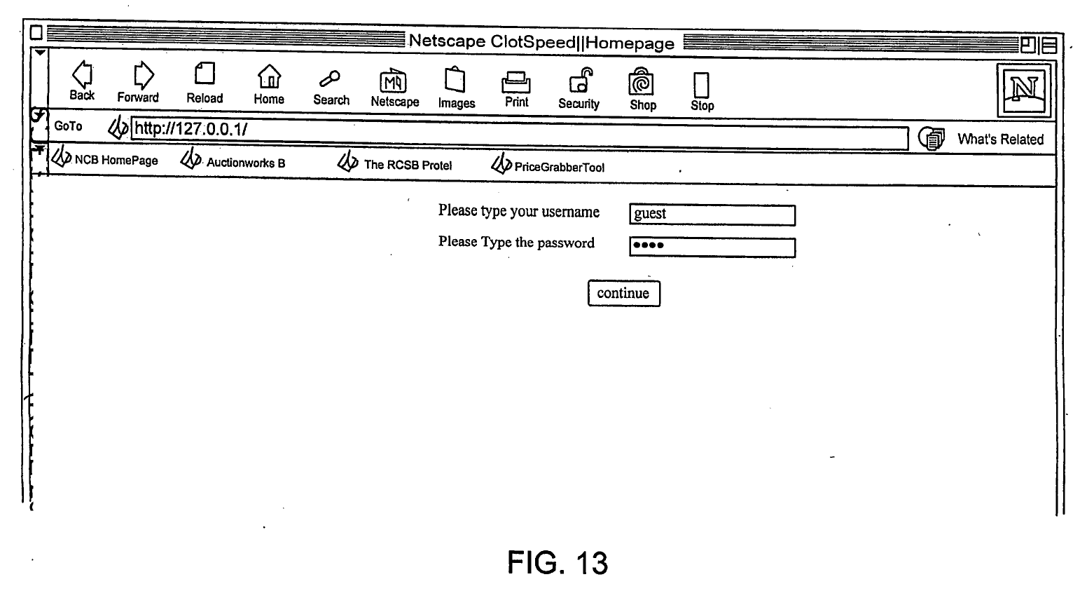

The screenshots of FIGS. 13 to 31 illustrate graphical windows displayed to a user that were used in one practical embodiment of a program written in accord with the aforesaid description.

FIG. 13 is a graphical window displayed to a user showing a login prompt for access to a modeling server. A user entered a user name and password in order to login to the database of the system.

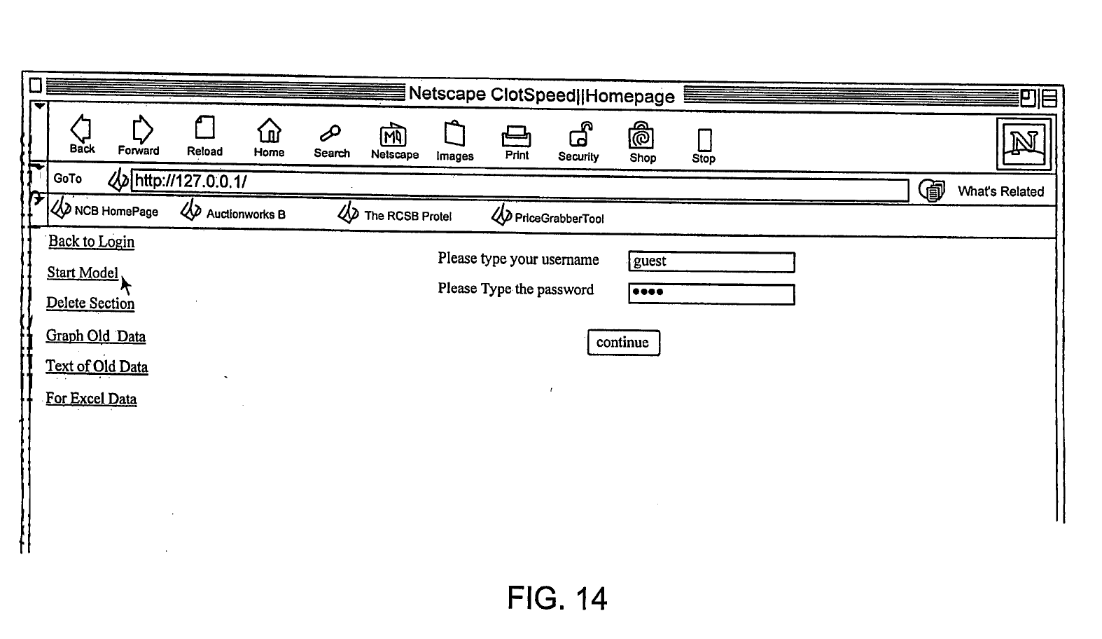

FIG. 14 is a graphical window displayed to a user showing a login prompt for a database. In this embodiment, once the user's password was accepted, the software program displayed only the menu at the left.

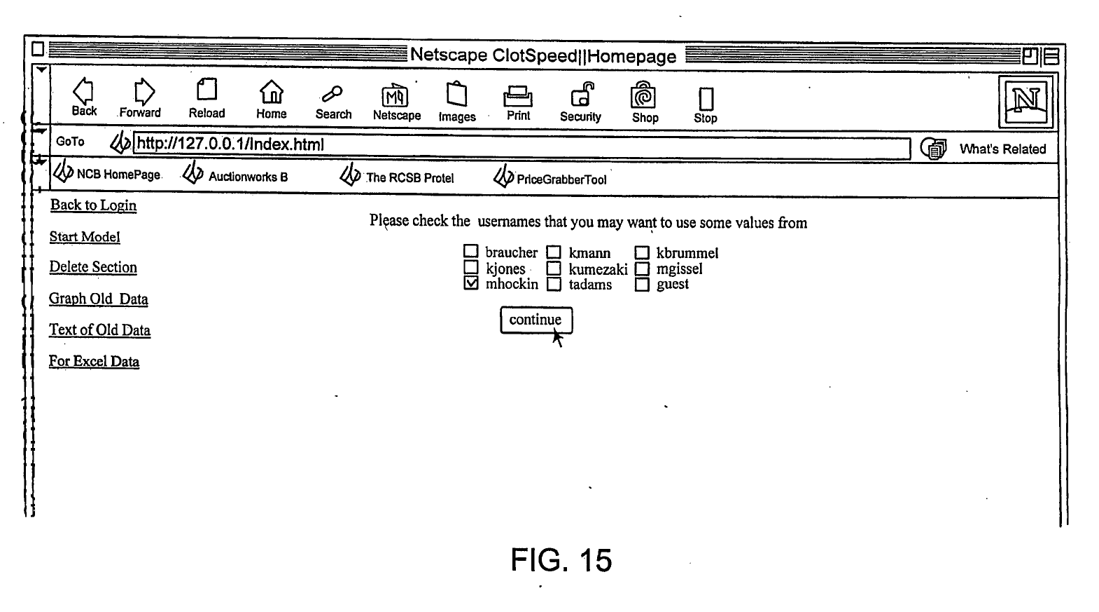

FIG. 15 is a graphical window displayed to a user showing a list of other users from which a user can select to use equations, species and/or rate constants.

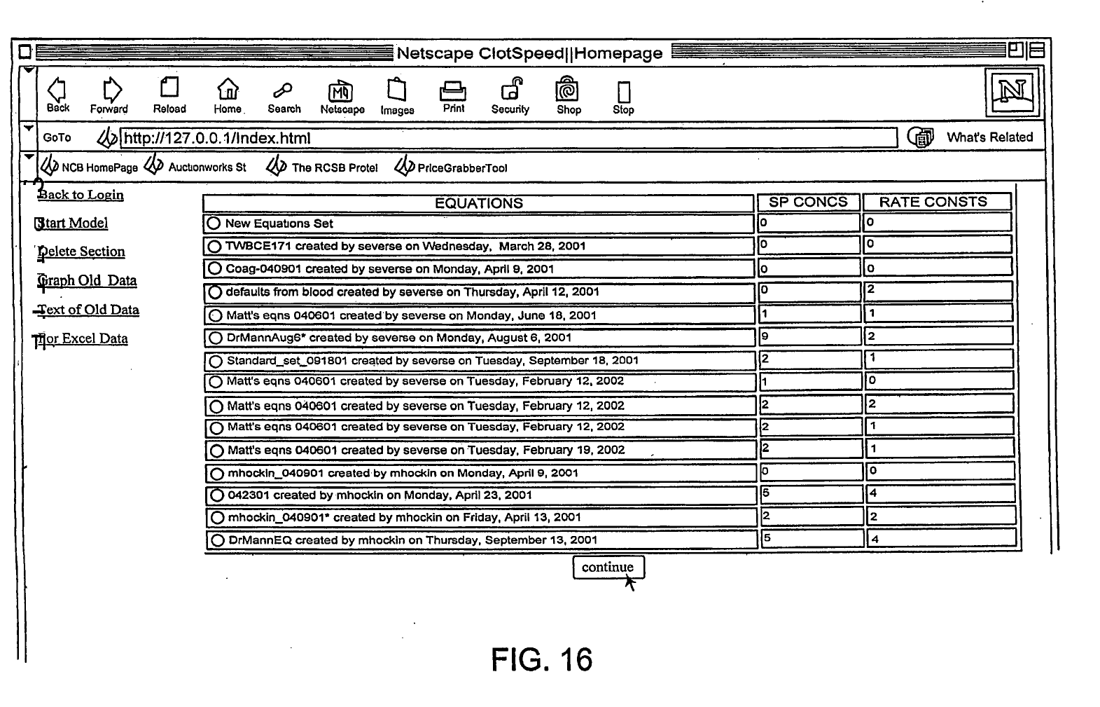

FIG. 16 is a graphical window displayed to a user showing a list of stored equations generated from the selections of FIG. 15.

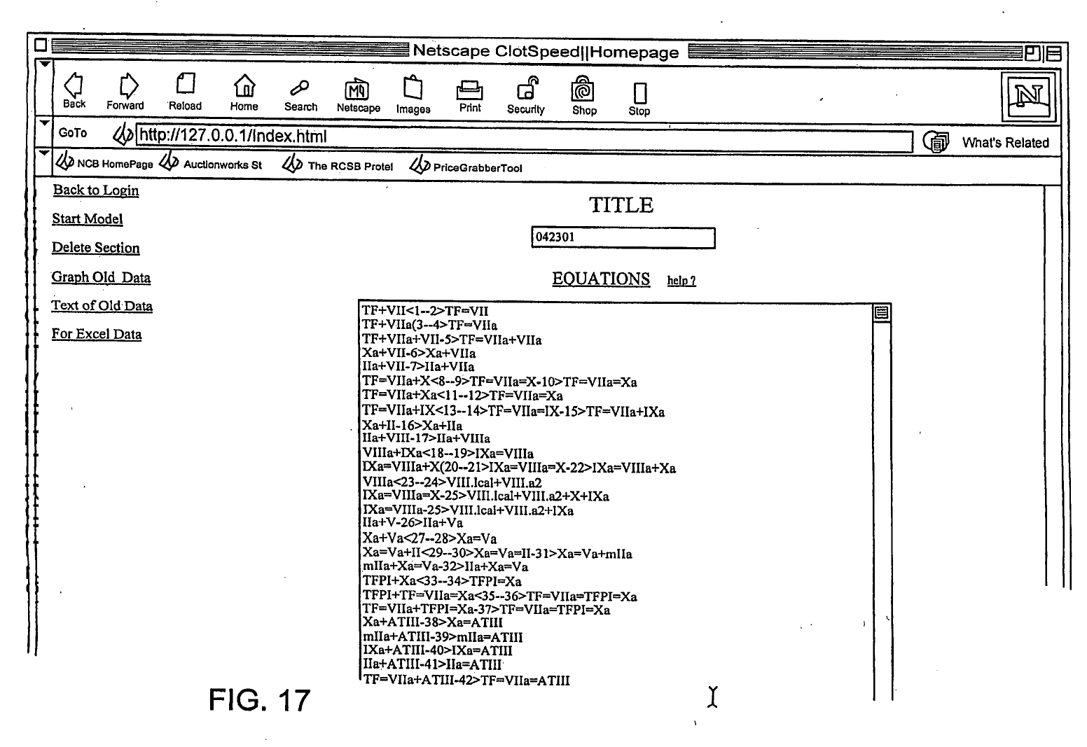

FIG. 17 is a graphical window displayed to a user showing a list of equations used in a run. This list of equations could have been, in one example, be a list from a prior run. In another example, the list of equations could have been entered by a user.

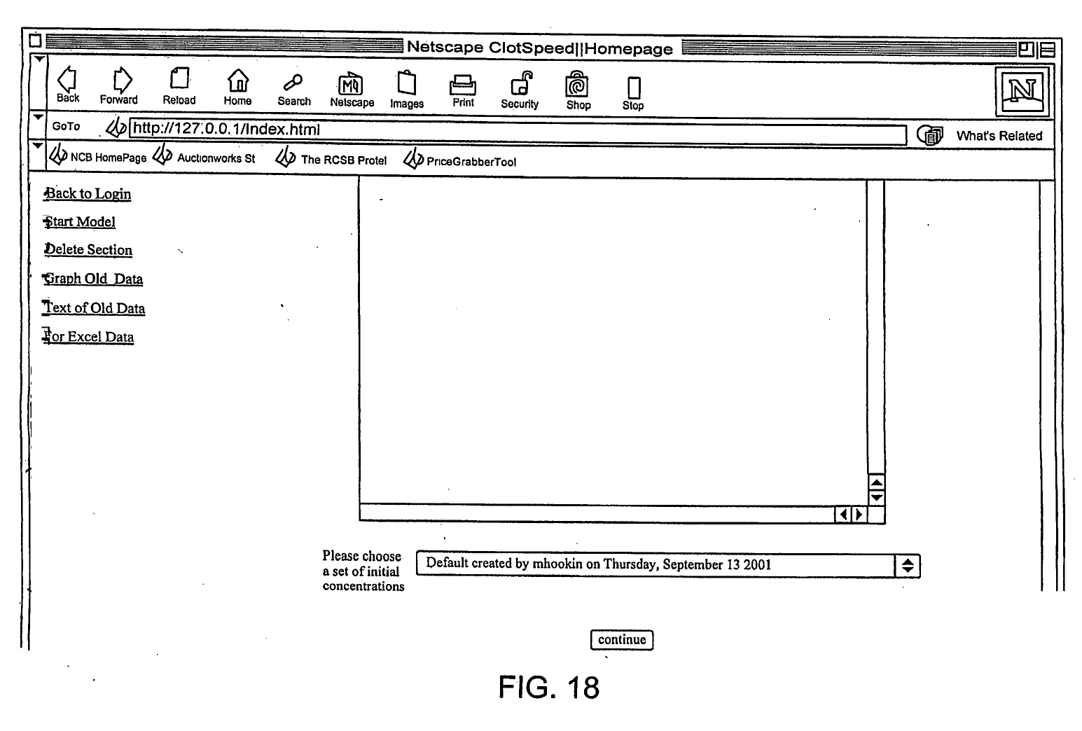

FIG. 18 is the lower half of the graphical window of FIG. 17 displayed to a user showing a text box for the entry of equations and a pull down menu from which species concentrations from prior data can be selected. It will be appreciated that the graphical window of FIG. 15 permitted the user a selection of initial species concentrations from prior data.

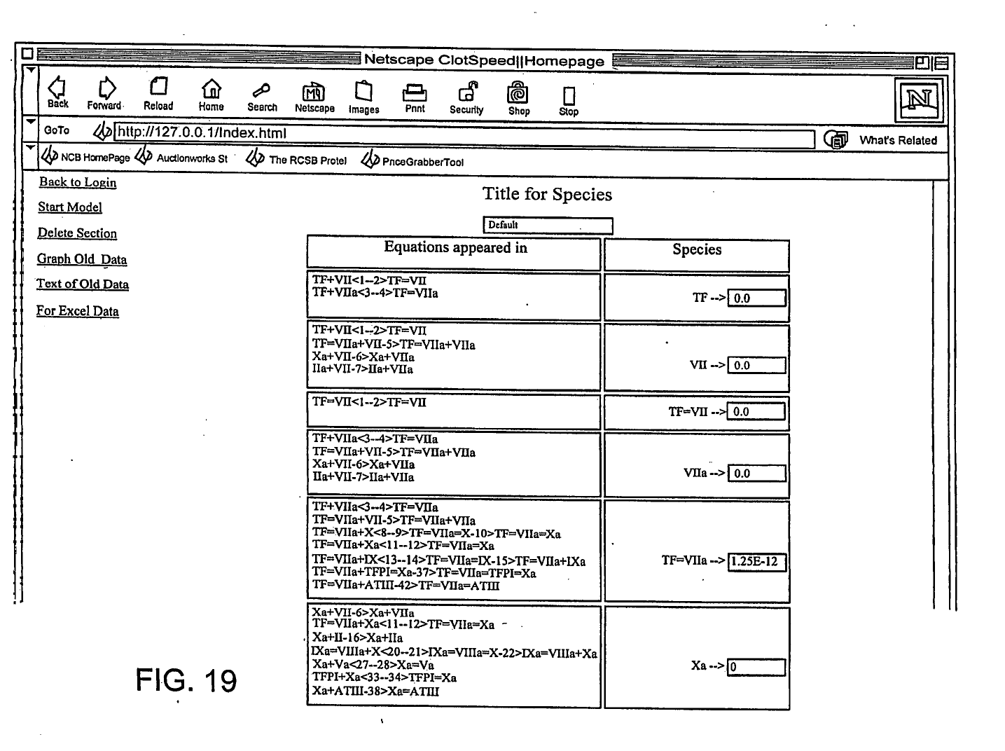

FIG. 19 is a graphical window displayed to a user allowing input of a species title and initial species concentrations. The user supplied this title in order to identify the species concentrations for later selection. It will be appreciated that the values inserted could have been new or chosen from the database.

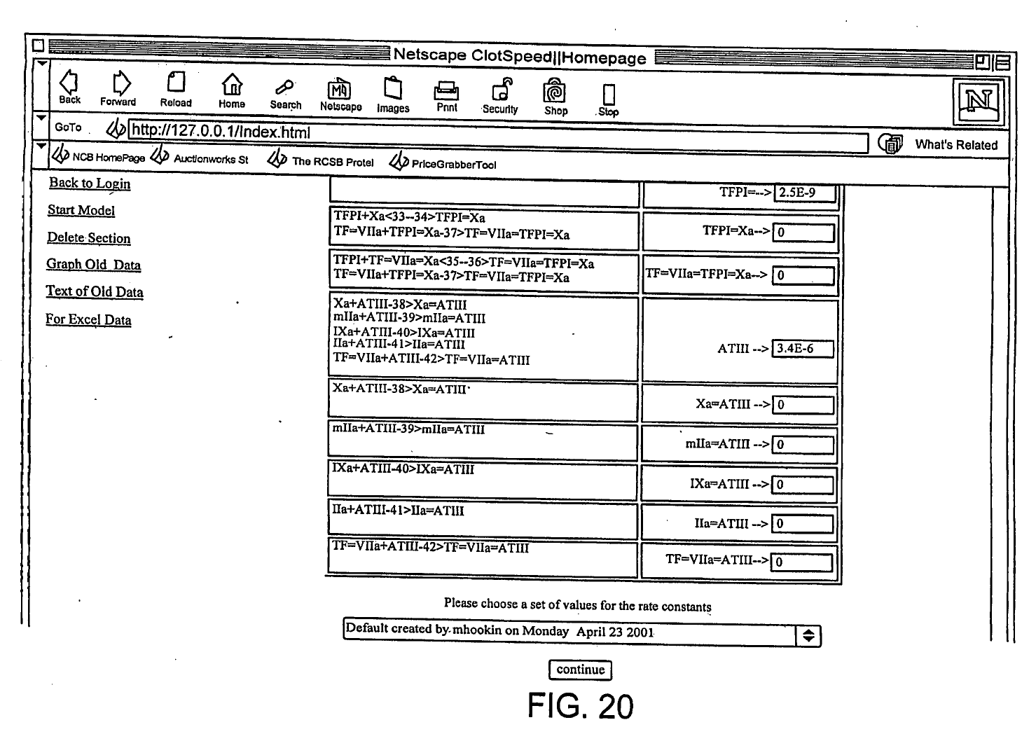

FIG. 20 is the lower half of the graphical window of FIG. 19 wherein the user was prompted to select rate constants from prior data. It will be appreciated that the graphical window of FIG. 15 permitted the user selection of rate constants from prior data.

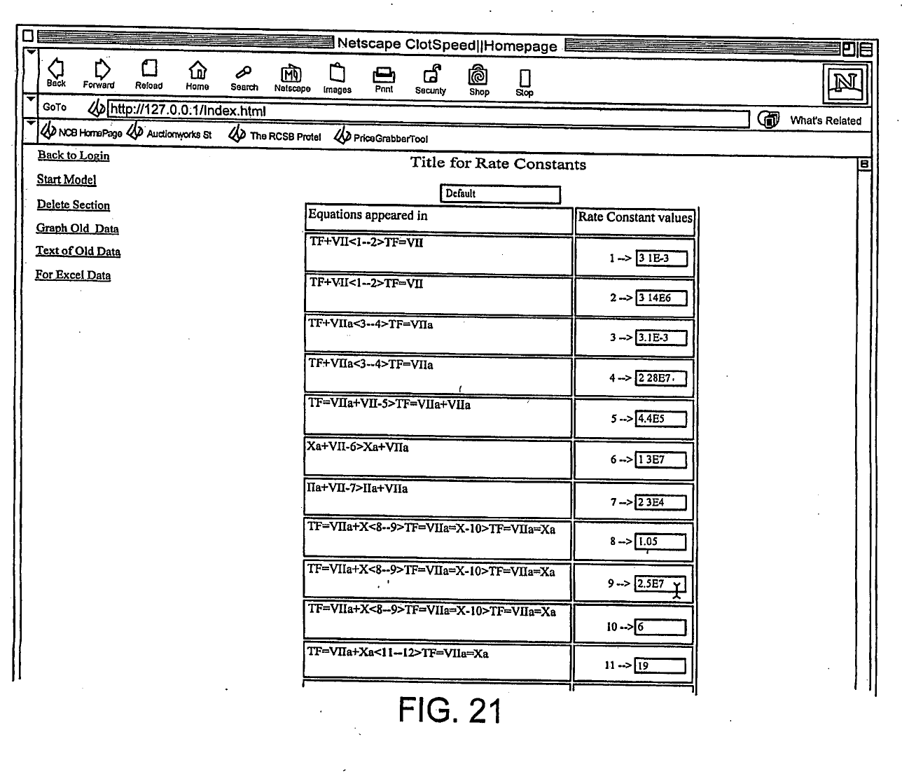

FIG. 21 is a graphical window displayed to a user showing rate constants inserted in a database for a run. It will be appreciated that the values could be fetched from the database or entered by the user.

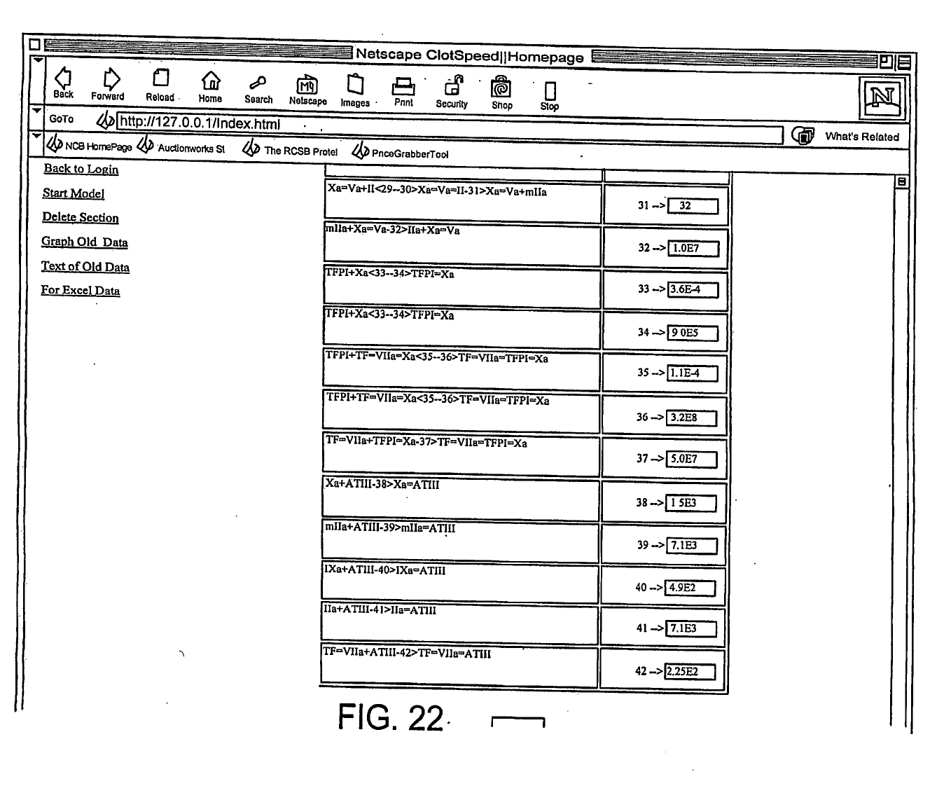

FIG. 22 is; the lower half of the graphical window of FIG. 21 wherein the user was prompted to enter rate constants for the equations into the database.

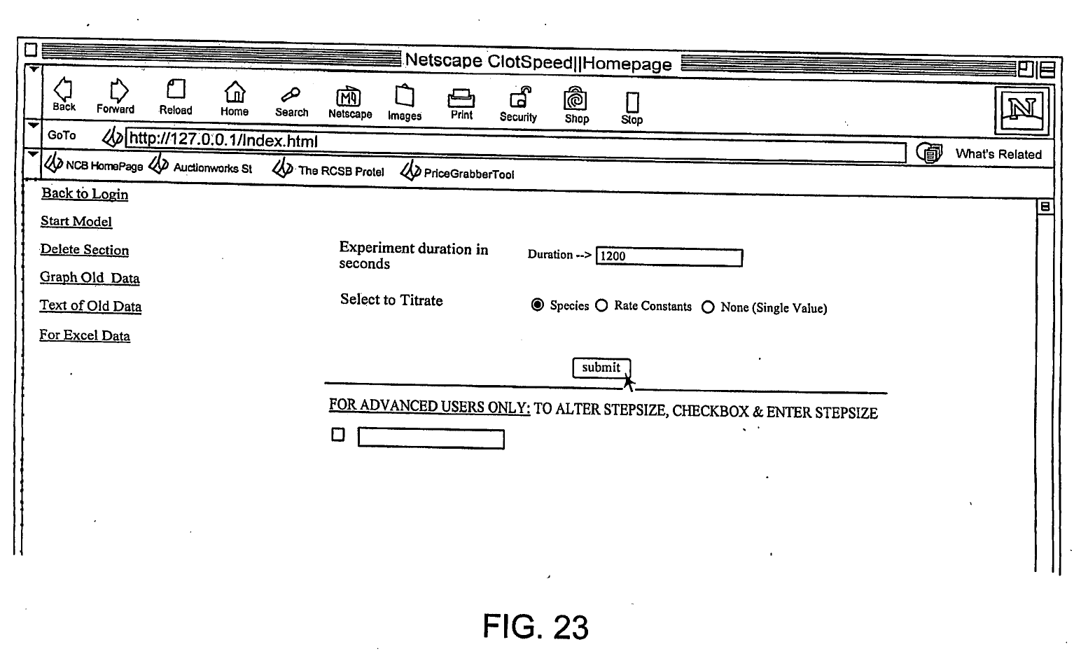

FIG. 23 is a graphical window displayed to a user showing a text box for the entry of an experimental duration and the selection of a titration experiment. It will be appreciated that a user could have selected no agent, in which case, no titration would have occurred.

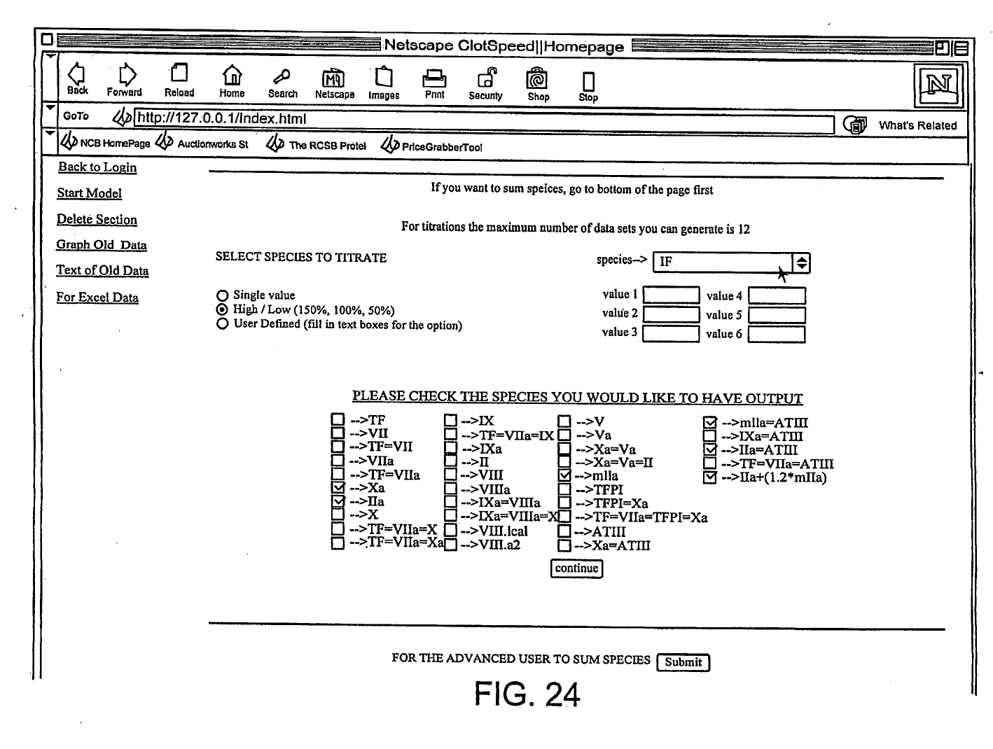

FIG. 24 is a graphical window displayed to a user showing a pull down menu with the selections available (species or rate constants) if a titration option had been selected in FIG. 23. It will be appreciated that, regardless of whether titration was selected or not, the checkboxes for output were made available.

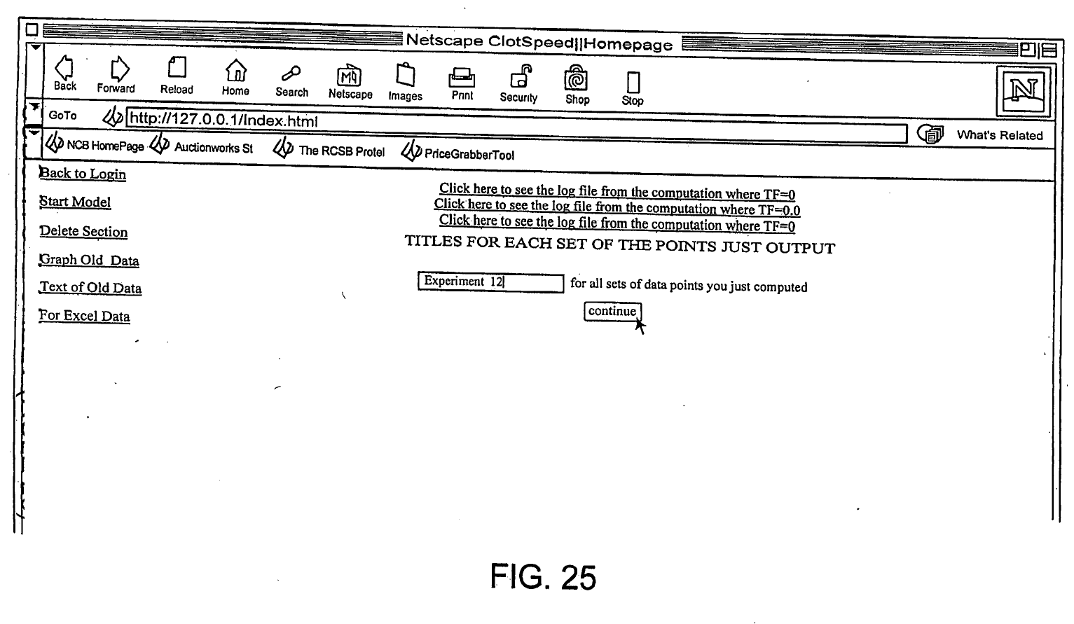

FIG. 25 is a graphical window displayed to a user permitting the entry of a title to an experiment thus allowing future selection of this information in other experiments. In one example, a user was permitted to select to use the same data for a second run, as illustrated in FIG. 16.

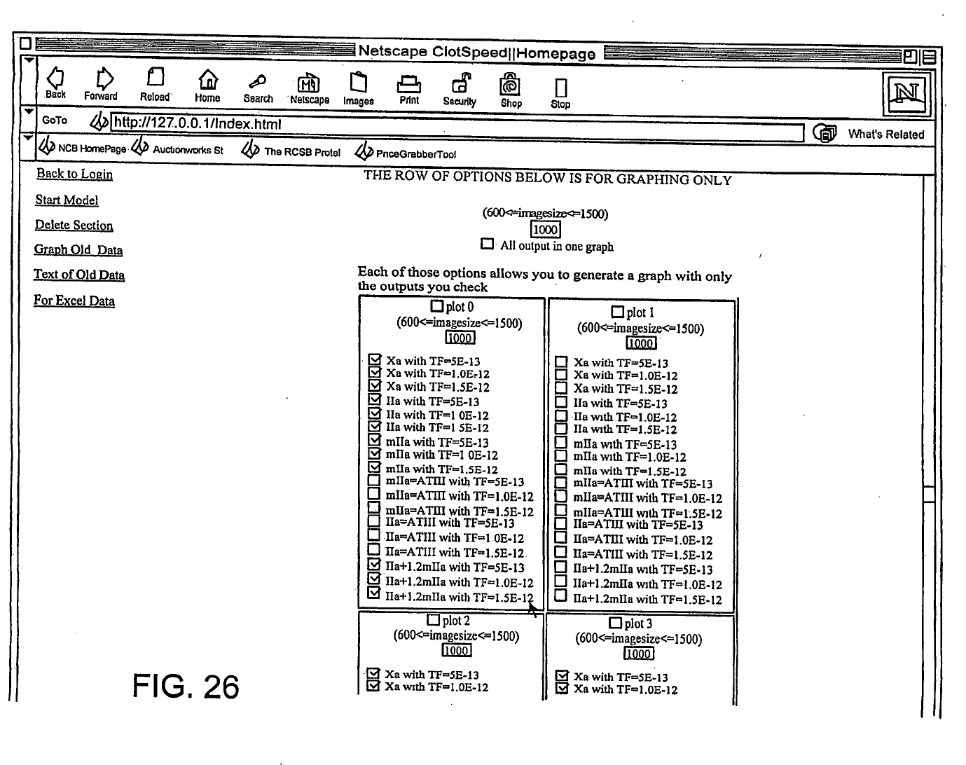

FIG. 26 is a graphical window displayed to a user showing a selection of data for a new graph.

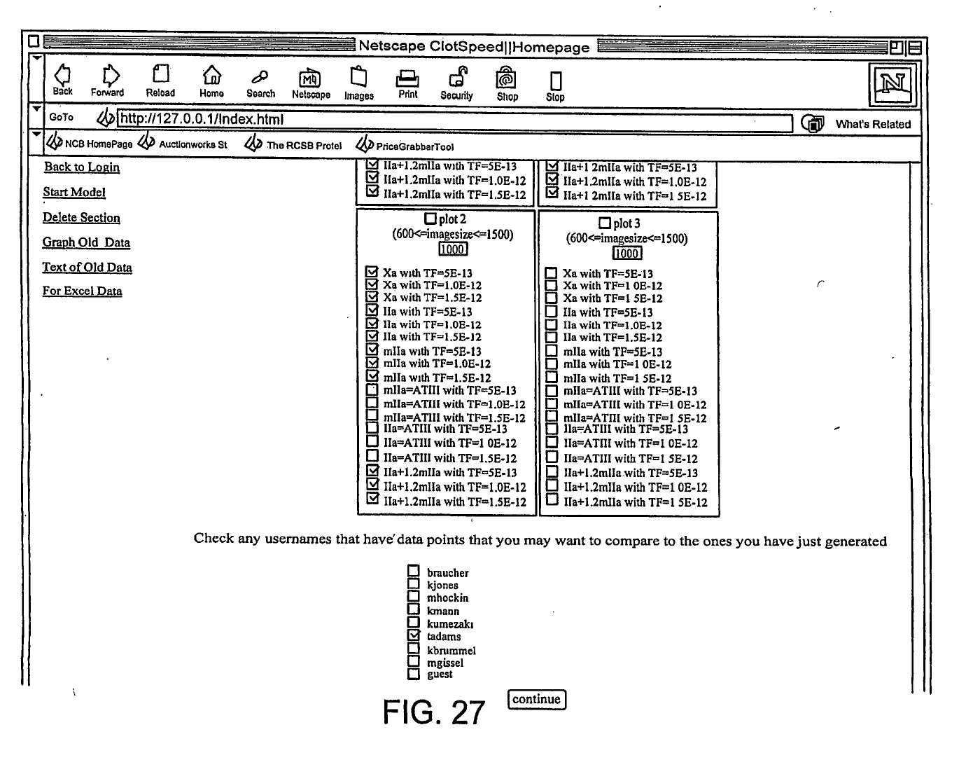

FIG. 27 is the lower half of the graphical window shown in FIG. 26 allowing a user to select data for a graph and to select other users's data in comparison.

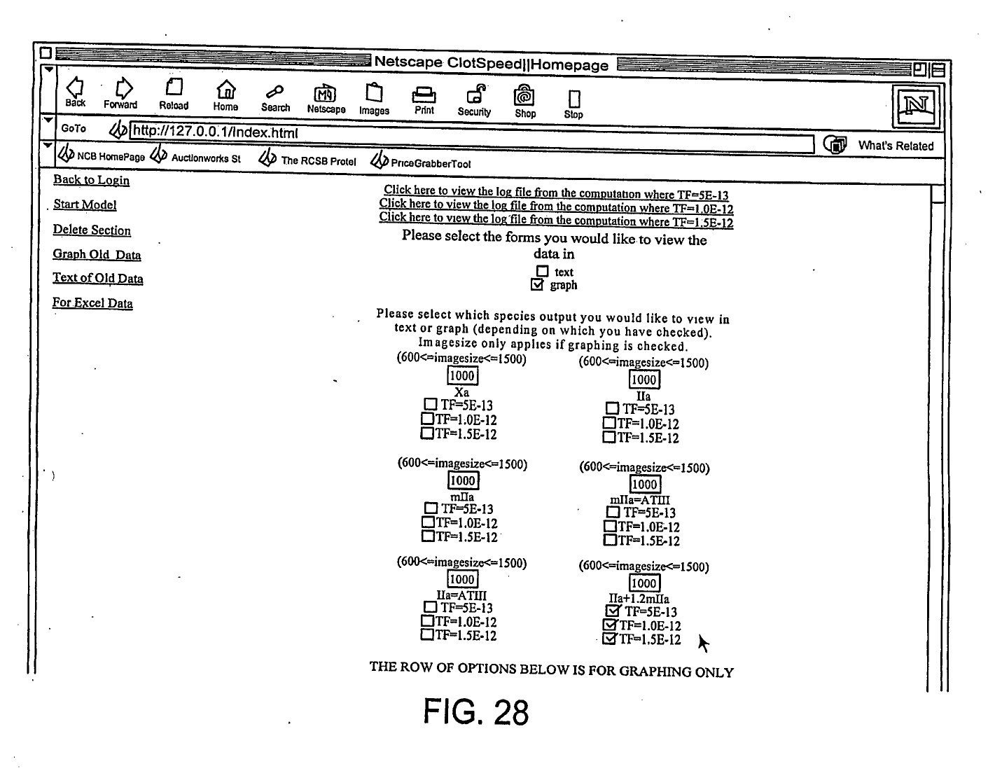

FIG. 28 is a graphical window displayed to a user showing a selection of output formats.

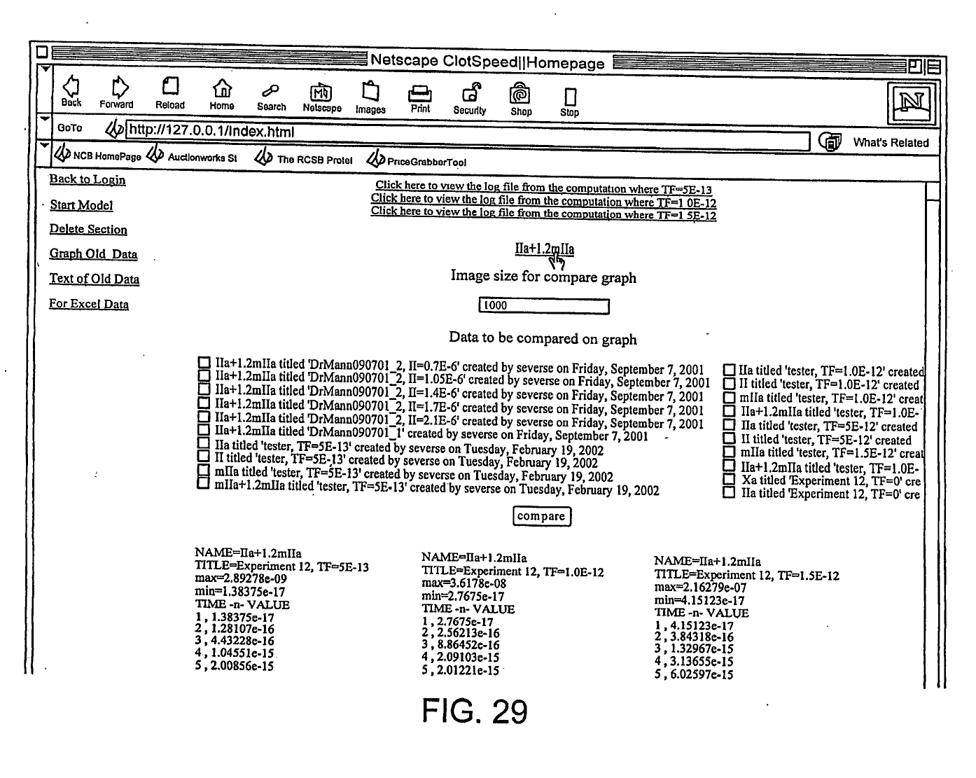

FIG. 29 is a graphical window displayed to a user showing a selection of image size for a graphical representation of data from a run. In the FIGURE, the hand is positioned to select an image size for the graphical representation of data. It will be further appreciated that a user could have selected prior data to be graphically displayed by checking all the boxes in the FIGURE.

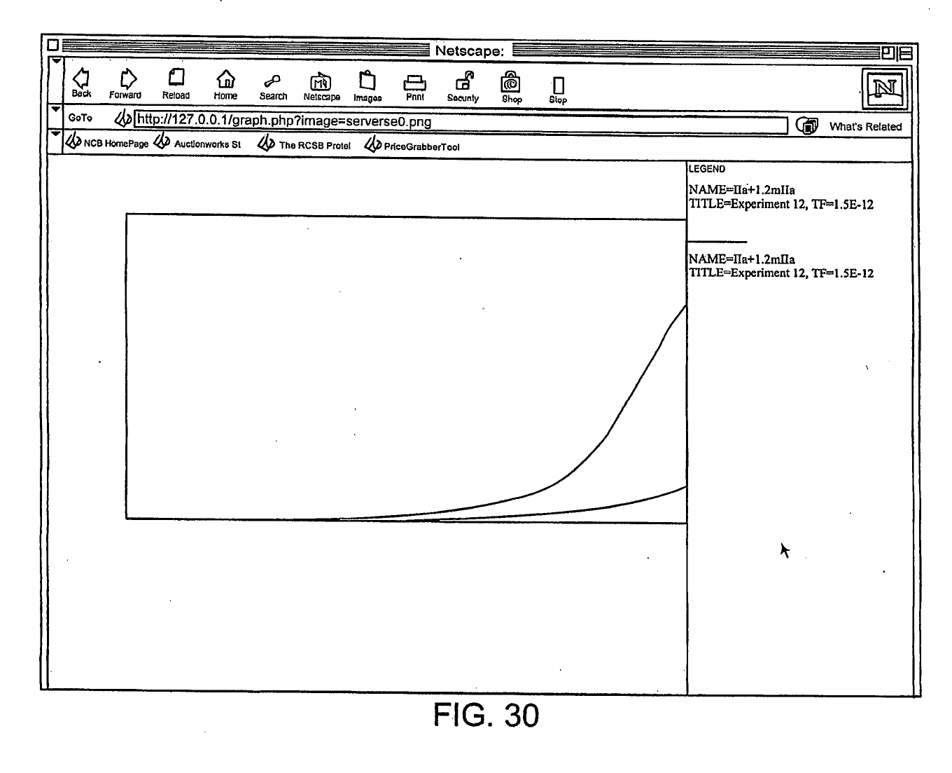

FIG. 30 is a graphical window displayed to a user showing a graphical representation of data from a run.

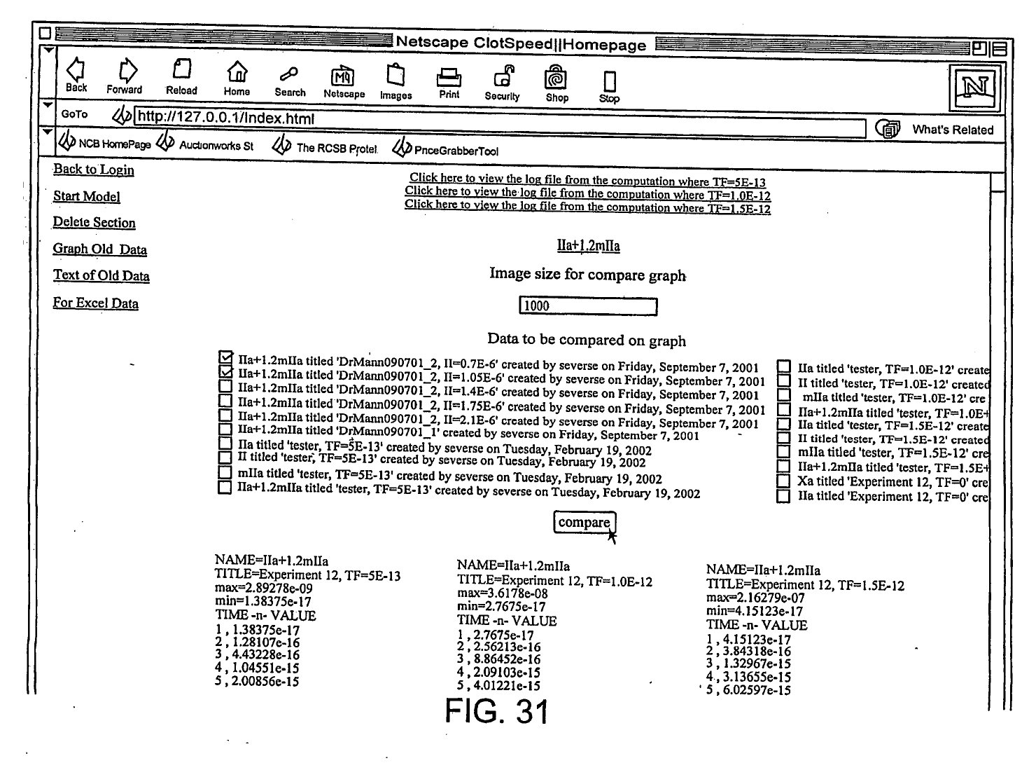

FIG. 31 is a graphical window displayed to a user showing a selection of other users date (note checked boxes) to be compared.

### EXAMPLES

The invention will now be further illustrated with reference to the following examples. It will be appreciated that what follows is by way of example only and that modifications to detail may be made while still falling within the scope of the invention.

### Example 1

### Analysis of Procoagulants in the Absence of Inhibition

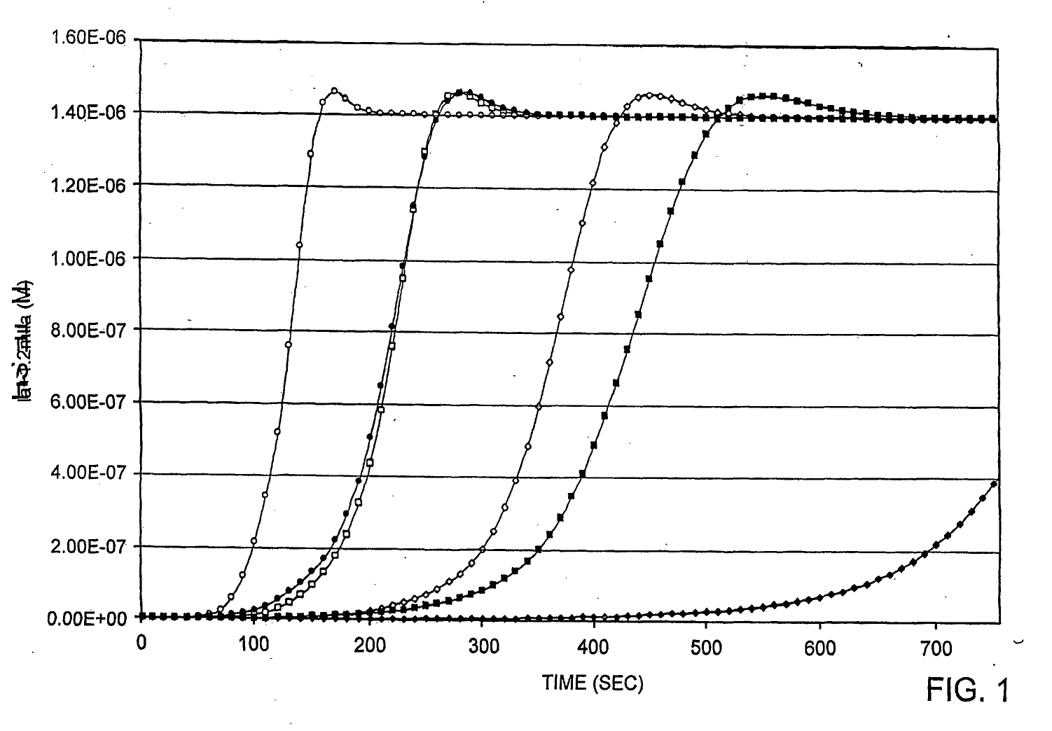

Simulations (FIG. 1, open symbols) were performed in the context of only the procoagulation model (Table 1, #1-19). Increasing TF concentrations (1, 5, 25 pM) result in reduction of the duration of the initiation phase which is arbitrarily defined as the time from introduction of TF necessary to generate 20 nM thrombin. The concentration of thrombin is represented as the activity measured using the synthetic substrate S-2238. The “bump” observed prior to the stable final value (1.4×10⁶ M) is a consequence of the 20% greater activity displayed toward this substrate by meizothrombin (Jones and Mann, 1994, J. Biol. Chem. 269, 23367-23373; Doyle and Mann, 1990, J. Biol. Chem. 265, 10693-10701). Over the range of TF illustrated in the absence of TFPI (open symbols) the maximum rate of thrombin production varied approximately five fold. In the present model the initial activation of prothrombin occurs; by factor Xa-membrane and the initial activation of factor V occurs by thrombin generated from the former reaction. The elimination of the factor Xa-PCPS activation of prothrombin (Table 2, k₁₆=0) under the set of equations detailed in Table 1 (omit #20-27), generated simulations with no thrombin production.

The factor VIIIa decay term based upon the empirically measured A₂ dissociation rate (Table 1, #13-15) (Fay, et al., 1996, J. Biol. Chem. 271, 6027-6032) increases the sensitivity of the reaction to reductions in factored concentration. The most notable characteristic of the procoagulants-alone data, also observed in empirical studies (van't Veer and Mann, 1997, J. Biol. Chem. 272, 4367 4377, Lawson, et al., 1994, J. Biol. Chem. 269, 23357-23366), is its biphasic behavior, a lag or initiation phase followed by a propagation phase. The results obtained here are similar to those reported by van't Veer and Mann 1997 (J. Biol. Chem. 272, 4367-4377) for similar procoagulant mixtures in the absence of inhibitors. However, in contrast to the results in FIG. 1, the published studies of van't Veer and Mann were conducted in the absence of factor VII and with reaction initiation conducted by the addition of preformed factor VIIa-TF complex to the reaction system. Modeling experiments conducted under the explicit conditions described by van't Veer and Mann are nearly identical to those published (Jones and Mann, 1994, J. Biol. Chem. 269, 23367-23373).

At fixed TF concentration, increasing the concentration of factor Vila shortens the duration of the initiation phase in a saturable manner. As observed in empirical experiments, the dependence of the duration of the lag phase appears in the VII/VIIa ratio, rather than the absolute factor VIIa concentration. This dependence is a function of the competitive binding equilibria between factor VII and factor VIIa and holds under conditions in which the binding isotherm (Table 1, #1-2) is at least partially saturated (i.e. >1 nM total VII/VIIa), given by the ratio of the rate constants k₁/k₂. These results illustrate the sensitivity of TF induced coagulation to levels of the TF=VIIa complex and thus the rate of factor Xa production at any point in the cascade as observed in the “wet” experiments by van't Veer and Mann 1997 (J. Biol. Chem. 272, 4367-4377).

When TFPI at 2.5 nM is added to the titrations observed in FIG. 1 (closed symbols), a significant extension of the initiation phase of the reaction is observed with only a relatively small effect on the propagation rate. This observation is in agreement with empirical experiments published by van't Veer et. al., which illustrated that the major effect of TFPI is in prolonging the initiation phase of the reaction.

### Example 2

### Effect of the Combination of AT-III and TFPI on TF Initiated Thrombin Formation

The addition of AT-III to the procoagulant reaction requires rate equations for IIa, mIIa, factor Xa, factor IXa and TF-factor VIIa complexation with this inhibitor (Table 1, #23-27). When compared to the procoagulant-alone system, simulations including AT-III exhibit bell shaped curves for thrombin generation at all TF concentrations tested. When challenged with 25 pM TF in the presence of 3.4 μM

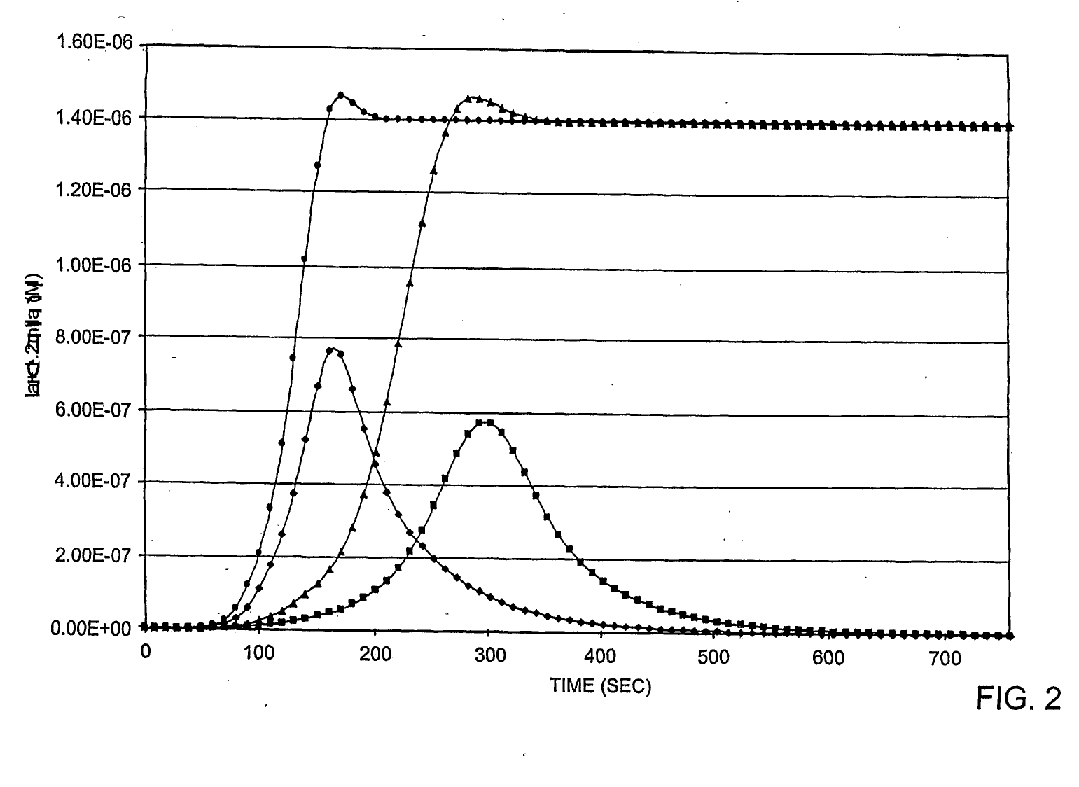

AT-III (FIG. 2 diamonds), thrombin production is slightly delayed, is at a maximum near 150. seconds, subsequently decreases and is nearly consumed by 400 seconds. Reactions with TFPI, in the absence of AT-III, 25 pM TF (triangles) yield maximal rates of thrombin production at ˜200 seconds and quantitative activation by 300 seconds. As observed in “wet” experiments AT-III does little to alter the duration of the initiation phase or the maximum rate of thrombin formation while TFPI (Table 1, #20-22) results in extension of the initiation phase of thrombin generation (FIG. 2 triangles). The addition of both TFPI and AT-III to the reaction system results in equivalent total thrombin generation at 25 pM TF (squares) but the reaction is significantly delayed.

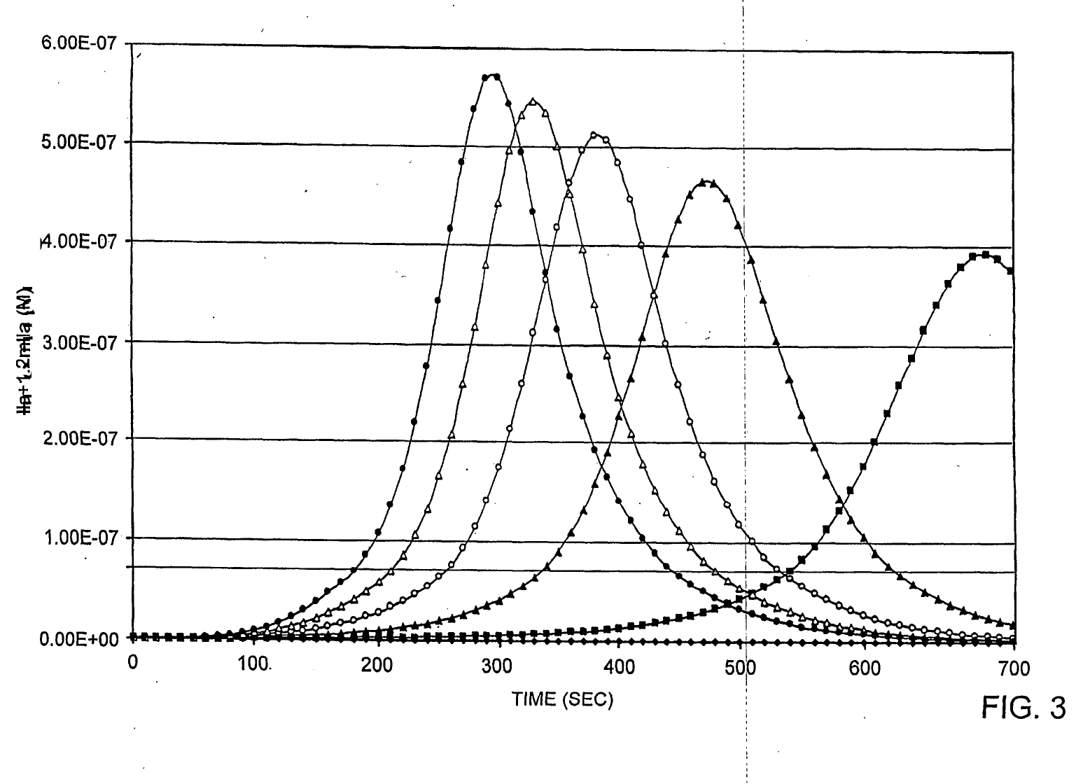

FIG. 3 illustrates a TF titration (1-25 pM) of the procoagulant system complemented with 2.5 nM TFPI and 3.4 μM AT-III. Active thrombin is plotted vs. time. The data illustrate that between the TF concentrations of S pM (filled squares) and 1 pM (filled diamonds) there is virtual attenuation of the thrombin formation response i.e. a threshold in this reaction. This synergistic effect of the two inhibitors acting in concert is similar to the empirically observed synergy observed in “wet” chemistry experiments reported by van't Veer and Mann 1997 (J. Biol. Chem. 272, 4367-4377), when these two inhibitors were combined with all procoagulants in TF initiated reactions.

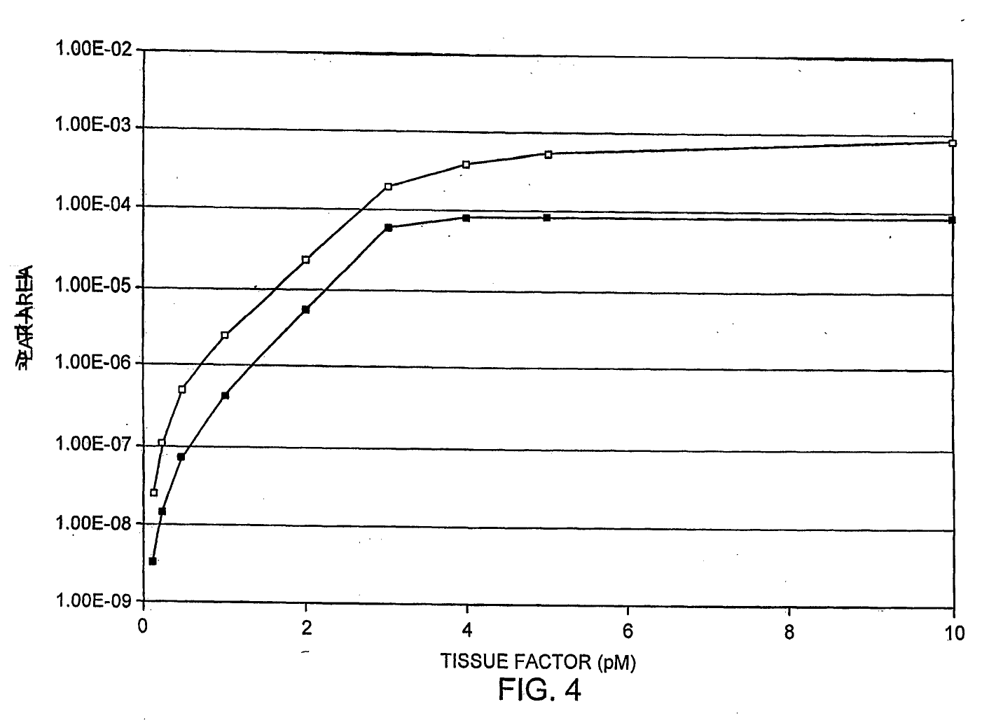

A quantitative interpretation of the data of FIG. 3 in terms of the total amount of thrombin produced over a 700 second time interval is illustrated by an exponential plot of thrombin peak area (IIa•seconds) vs. TF concentration in FIG. 4. For the concentration range from 3 to 10 pM, and extending to 25 pM TF (data not shown) the total thrombin and active thrombin are unchanged. At TF concentrations below 3 pM, an exponential concentration dependence is observed with almost a thousand-fold decrease in active thrombin (filled symbols) between 3 pM and 1 pM.

Thus, the theoretical model mirrors the empirically observed effect of the two inhibitors, TFPI and AT-III acting in concert.

### Example 4

### Analysis of the Initiation Phase of the Procoagulant Response

Following complexation of factor VIIa with TF-PCPS (Table 1 # 2), the initiation phase begins with the activation of factor IX and factor X to their respective enzyme products (Table 1 #6, 8). As noted, the duration of this initiation phase is largely a consequence of factor VIIa and TF and regulation by factor VII and TFPI (Table 1 #1, 2, 21, 22). The factor Xa generated initially by the factor VIIa-TF complex (Table 1 # 6, 7) activates a small amount of prothrombin to thrombin (Table 1 # 9). That thrombin begins the process of catalyst building by activating factor V and factor VIII (Table 1 # 10, 16).

Although factor Xa-PCPS has the capacity to activate factor V (Foster, et al., 1983, J. Biol. Chem. 258, 13970-13977), empirical data (Butenas, et al., 1997, supra) shows conclusively that thrombin is the essential early activator in “wet” chemical experiments. Thus, crucial to the initiation phase is the activation of some prothrombin to thrombin by factor Xa-PCPS exclusive of factor Va. This initial catalyst generates the thrombin, which initially activates some factor V, and factor VIII to their respective cofactor (factor Va, factor VIIIa) products.

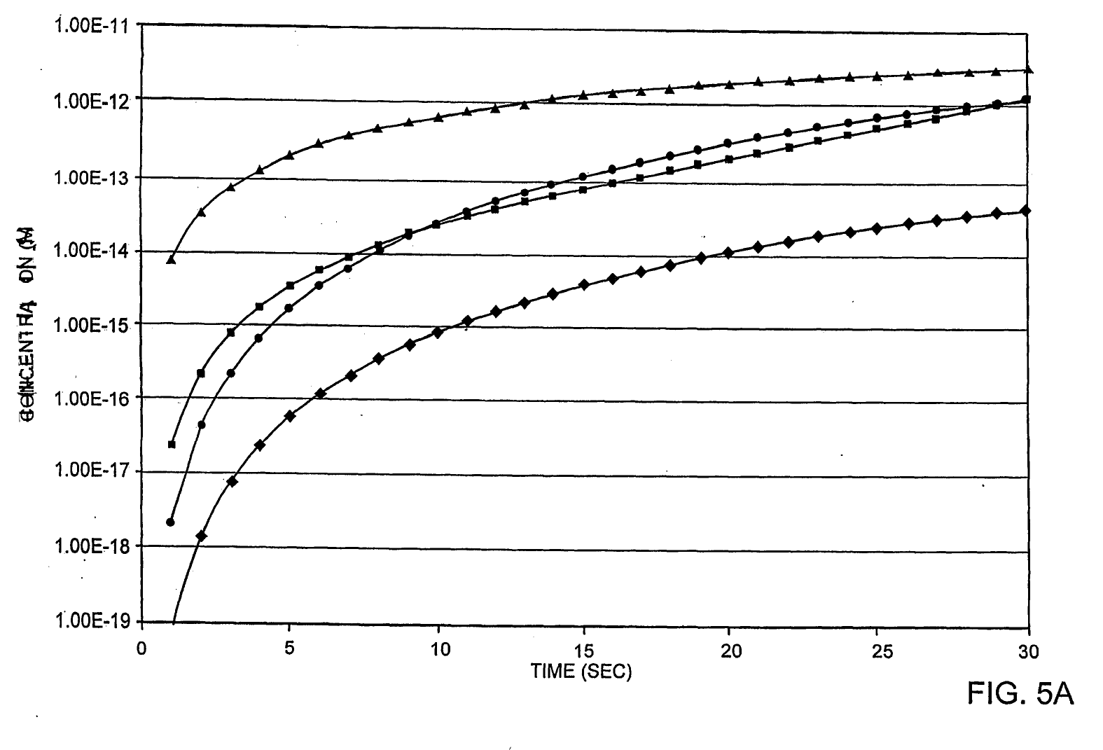

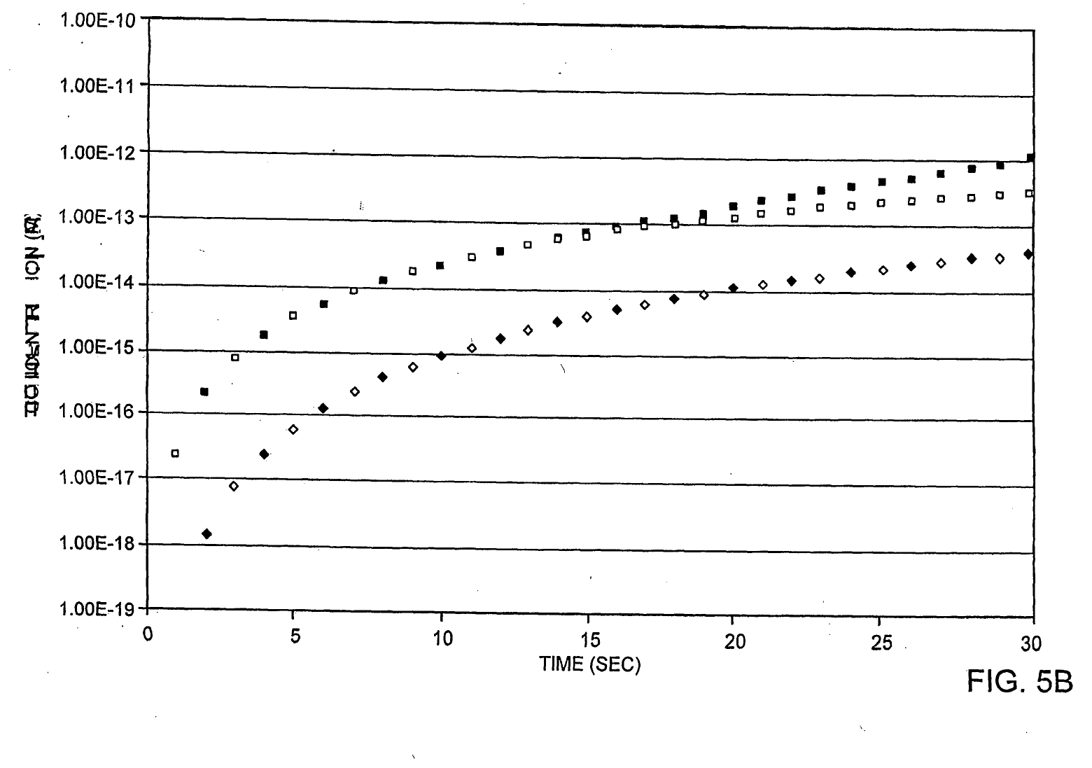

FIG. 5A illustrates a simulation of a reaction initiated with 5 pM TF during the first thirty seconds. Displayed, on an exponential scale, are the concentrations of active thrombin (squares), factor Xa (triangles), factor Va (circles) and factor VIIIa (diamonds) in the reaction as a function of time. The data are plotted on an exponential vertical axis, which reflects the diminishingly small concentrations of products in the early part of the reaction.

The point at which one observes the initial contribution of factor Va to the generation of thrombin is shown graphically in FIG. 5B, which illustrates a comparison of thrombin (squares) and factor VIIIa (circles) formation over the initial 30seconds in the presence (filled symbols) and absence (open symbols) of factor V. This FIGURE illustrates that during the first twelve seconds of the reaction, thrombin is produced by factor Xa; independent of a factor Va contribution. Subsequently, after twelve seconds, the feedback activation of factor V permits formation of prothrombinase, which provides increased thrombin levels (filled squares). This may be discerned by the derivation between the factor V replete (closed symbols) and factor V deficient (open symbols) reactions at approximately the 15-second time points. It should also be noted that factor VIII activation (diamonds) is dominated by thrombin generated by factor Xa-PCPS during this interval, as the presence of factor V has no influence on factor VIIIa formation (filled and open diamonds).

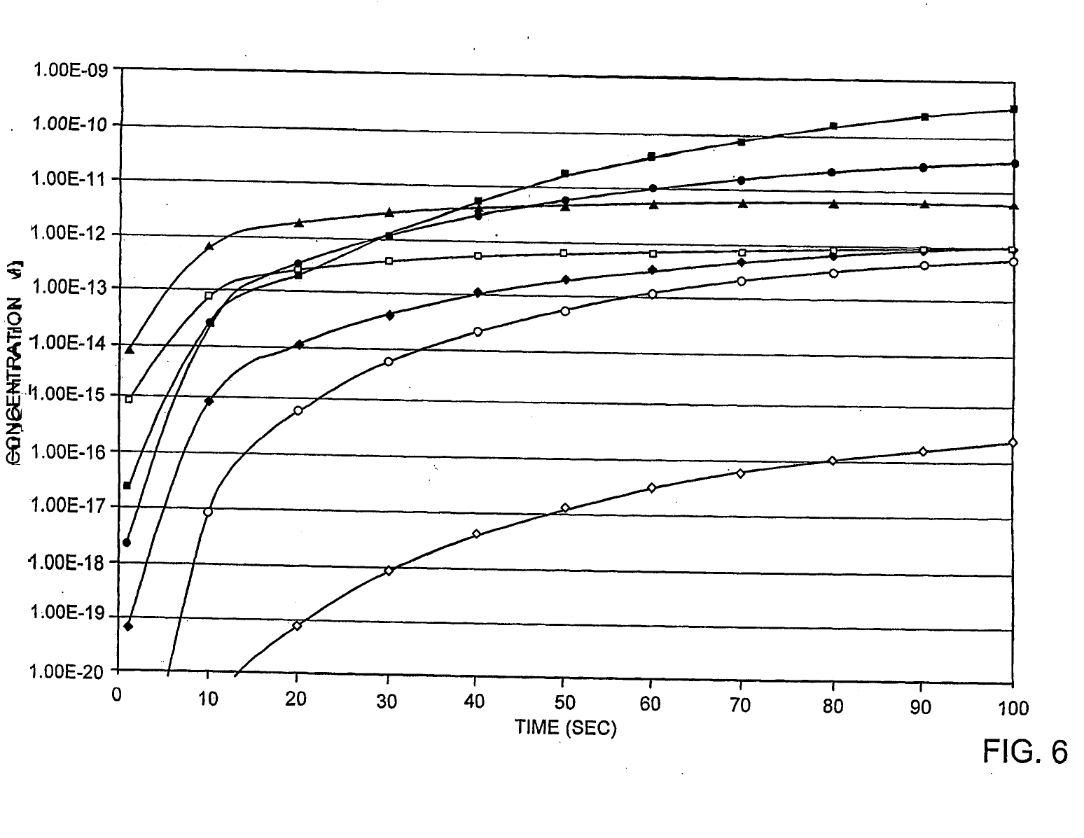

As the active cofactors are generated, the concentrations of prothrombinase and intrinsic factor Xase are rapidly increased. FIG. 6 illustrates the first 100 seconds of the reaction initiated by 5 pM TF. By 100 seconds, the factor Va concentration (filled circles) is ˜50 pM while factor VIIIa concentrations (filled diamonds) are ˜1 pM. It should be noted here that the factor Xa concentrations (filled triangles) is the limiting component for prothrombinase catalyst (open circles) formation which is ˜0.8 pM at 100 sec. In contrast the intrinsic factor Xase (open diamonds) at 100 seconds (˜0.3 fM) is governed by near equivalent concentrations of factor VIIa (filled diamonds) (˜1.0 pM) and factor IXa (open squares) (˜1.0 pM). Thus, the Kd for the intrinsic factor Xase plays a major role in regulating the total catalyst concentration to ˜0.3 fM.

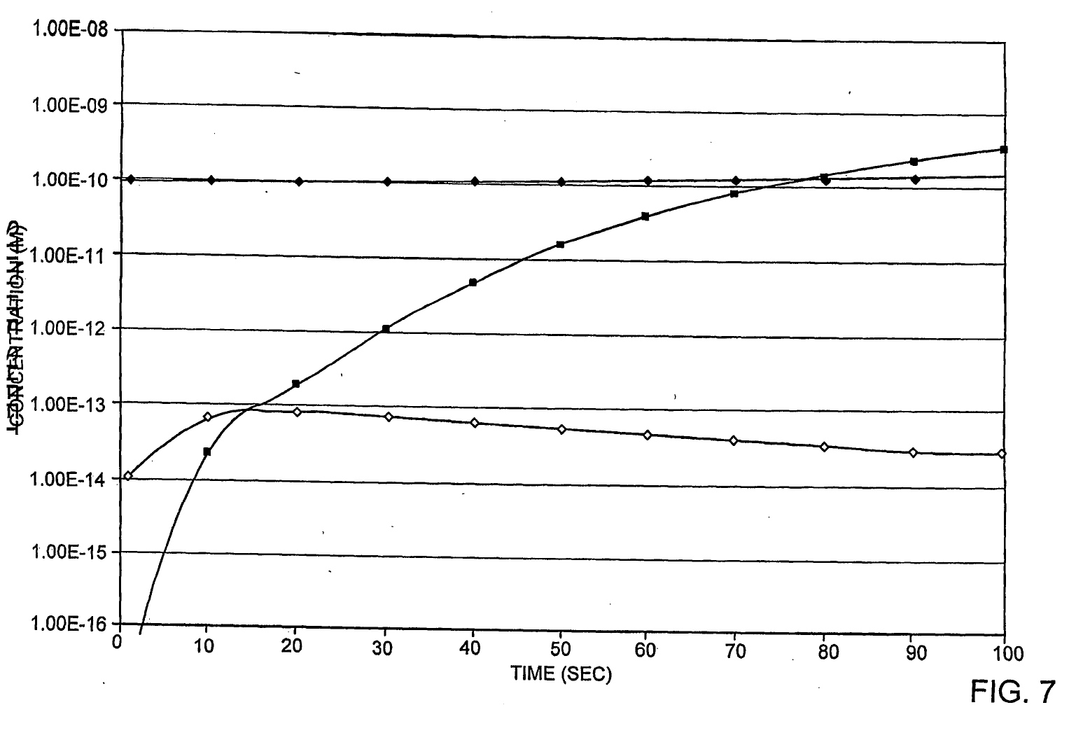

At the 100 sec interval (FIG. 7), it can be seen that the original factor VIIa concentration (filled diamonds) (˜0.2 nM) is only slightly increased by thrombin feedback activation (Table 1, #5) and the concentration of extrinsic factor Xase (˜4.0 fM) (open diamonds) is declining due to the action of TFPI and AT-III. The small inflection should be noted in total active thrombin concentrations (filled squares) which occurs between ˜10 and 20 seconds. This discontinuity is associated with the beginning of the transition from factor Xa-membrane to prothrombinase activation of prothrombin. By 100 seconds, thrombin concentrations are approaching 0.5 nM.

### Example 5

### Analysis of the Propagation Phase of the Reaction

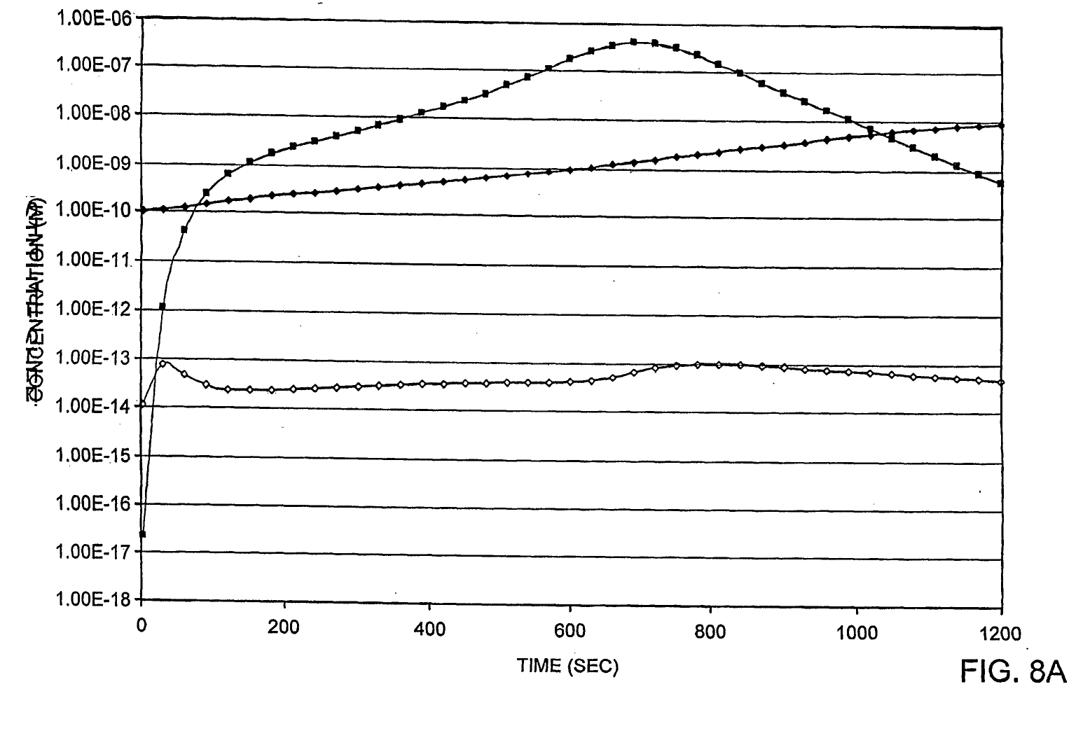

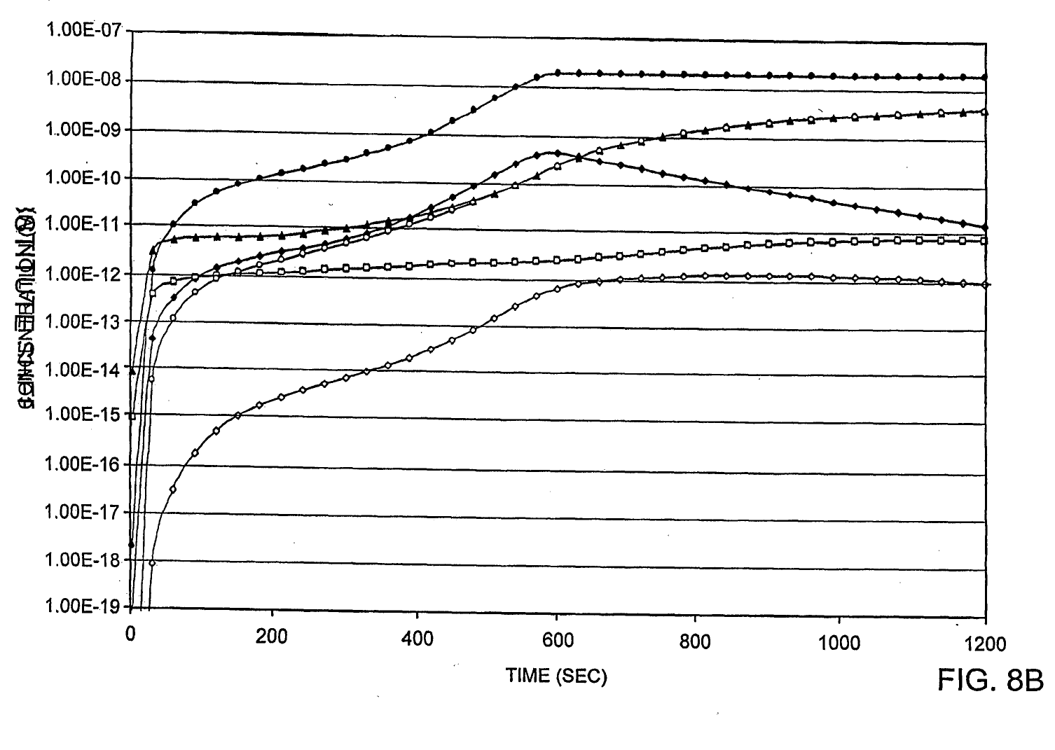

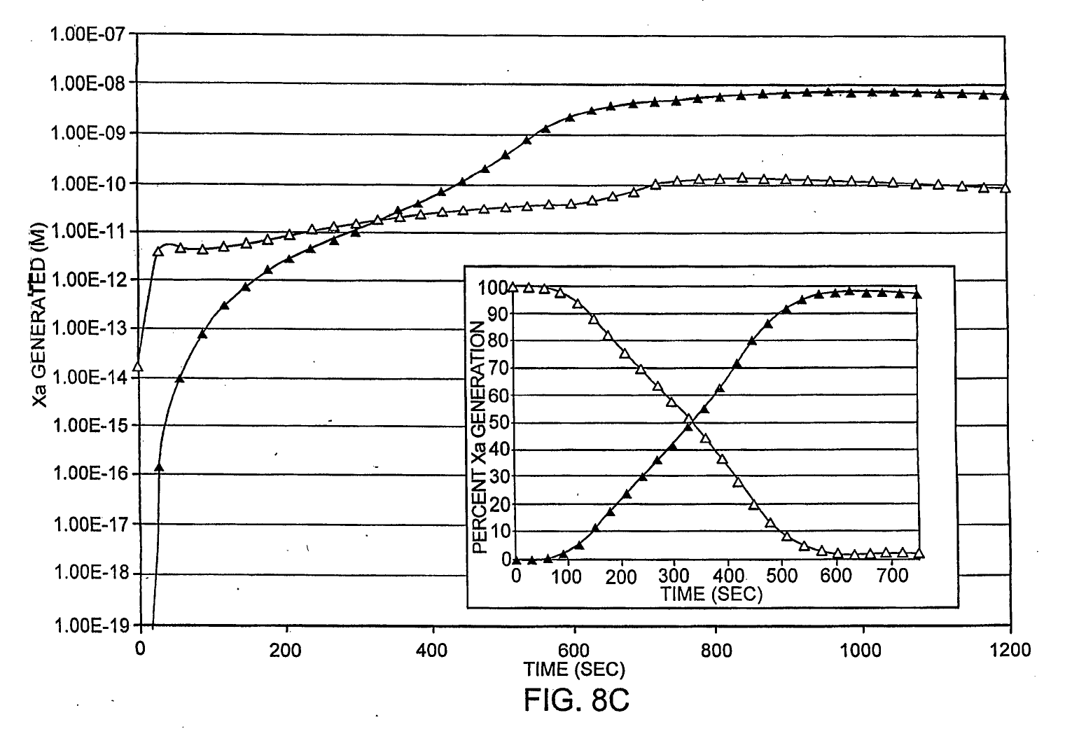

An expanded view of the reaction, which includes the propagation phase, is presented in FIGS. 8A and 8B. Active thrombin generation (FIG. 8A, filled squares) continues briskly until 700 seconds then begins to slow as-AT-III consumes thrombin and the catalysts that produce it. By 700 seconds, thrombin production and consumption are equivalent. If fibrinogen were present, clotting would have occurred at ˜400 seconds in this reaction (˜20 nM IIa) based upon evaluations of the similar reaction conducted in whole blood (Rand, et al., 1996, Blood 88, 3432-3445, Brummel, et al., 1999, J. Biol. Chem. 274, 22862-22870, Cawthern, et al., 1998, Blood. 91, 4581-4592). Factor VIIa generation (filled diamonds) continues to increase, leading to some largely irrelevant increases in the extent of the extrinsic factor Xase (open diamonds).

In FIG. 8b it can be seen that by 300 seconds, all of the factor V and factor VIII have been activated to factor Va (20 nM) (filled circles) and factor VIIIa (˜0.7 nM) (filled diamonds). Factor VIIIa declines in concentration noticeably beyond 600 seconds because the dissociation of the factor VIM A₂ domain (Table 1 # 13). The factor VIIIa dissociation process and the inhibition of factor IXa by AT-III leads to a progressive decline in the intrinsic factor Xase (open diamonds) beyond 600 seconds. Especially interesting is that over most of the time course, the prothrombinase concentration (open circles) is equivalent with the factor Xa concentration (filled triangles) generation curve illustrating the prominence of factor Xa as limiting component in the expression of prothrombinase, an observation initially made in the studies of Lawson et al. 1994, J. Biol. Chem. 269,23357-23366) and extended in subsequent studies of the TF induction of coagulation in whole blood (Rand, et al., 1996, Blood 88, 3432-3445).

The ultimate dominance of the intrinsic factor Xase (filled triangles) over the extrinsic factor Xase (open triangles) in factor Xa generation is illustrated in FIG. 8C which shows the concentrations of these two complexes over the time course of the reaction, while the inset to FIG. 8c displays the relative percentage of factor Xa delivered by the two catalytic complexes. Initially the extrinsic factor Xase (open triangles) is the major contributor to factor Xa generation because it is the catalyst at the highest concentration. However, by approximately 300 seconds the concentration of the extrinsic factor Xase is superseded by the concentration of the intrinsic factor Xase (closed triangles) whose catalytic properties are approximately 50 times more efficient than those of the factor VIIa-TF complex. As a consequence, even by 100 seconds factor Xa generation is dominated by the intrinsic factor Xase. These observations should be compared with the thrombin curve in FIG. 3 which illustrates that the maximum rate of accumulation of active thrombin occurs at ˜400 seconds while the peak of intrinsic factor Xase generation of factor Xa occurs at ˜600 seconds. FIG. 8C also illustrates the dominant role that thrombin plays in the activation of factor VII to factor VIIa; both catalysts peak between 600 and 700 seconds. The flattening of the two factor Xase catalyst propagation curves is a consequence of factor VIIIa dissociation and factor IXa and extrinsic factor Xase inhibition by AT-III.

### Example 6

### Study of the Termination Phase of the Reaction

Termination of the thrombin generating reaction is essential to eliminate ever-expanding thrombin generation and clot formation. Each event of catalyst formation is accompanied by a catalyst depletion mechanism. The clearest illustration of catalyst termination is the reduction in the concentration of thrombin under all model conditions. Thrombin inhibition is the ultimate result of complex formation with the stoichiometric inhibitor, AT-II. However, equally important are reductions of activities of the extrinsic and intrinsic factor Xase's, which contribute to further thrombin formation. AT-III, TFPI and factor VIIIa dissociation are the principle contributors to catalyst elimination in plasma coagulation. The role of TFPI is largely evident in the initiation phase of the reaction (FIGS. 1, 2). When integrated with AT-III the combination of inhibitors ensures that thrombin formation only takes place when sufficient initiating concentrations of TF are presented (FIGS. 4, 5). TFPI binds with several species including factor Xa (Table 1 #20) and the TF-factor Xa-factor VIIa product complex(Table 1 #21, 22). The limited concentration of TFPI plays a significant role by delaying initiation by inhibiting the factor Xa produced. The major role of AT-III in termination is related to nearly quantitative, general serpin inhibition.

The decay of factor VIIIa activity caused by dissociation of the A₂ domain also contributes to the termination phase of the reaction. Prior work has employed an abstract mathematical construction to accommodate the known reduction in factor VIIIa effectiveness to explain the slowing of factor X activation observed under empirical conditions (Jones and Mann, 1994, J. Biol. Chem. 269,23367-23373). In accord with the present invention, the rate constants for the established mechanism were used for factor VIIIa dissociation. This approach yields results that approximate the empirical observations which display attenuation of factor Xa activation rates.

The factor VIII-A₂ domain dissociation and reaction termination is essential to the regulation of the concentration of the procoagulant during the termination phase of the reaction. Factors V and VIII are completely converted to their active forms during the propagation phase and their depletion through APC (for factor Va) and subunit dissociation (for factor VIIIa) is enhanced when those cofactors are dissociated from their active enzyme complexes. Therefore it is necessary to keep factor Xa and factor IXa concentrations from expanding too rapidly (to allow cofactor dissociation). Factor VIIIa-A₂ dissociation is key to the decreased activity of the intrinsic factor Xase activity. Just as the propagation phase is controlled by the expanding concentration and function of the intrinsic Xase activity, the termination phase is controlled by a reversal of this process.

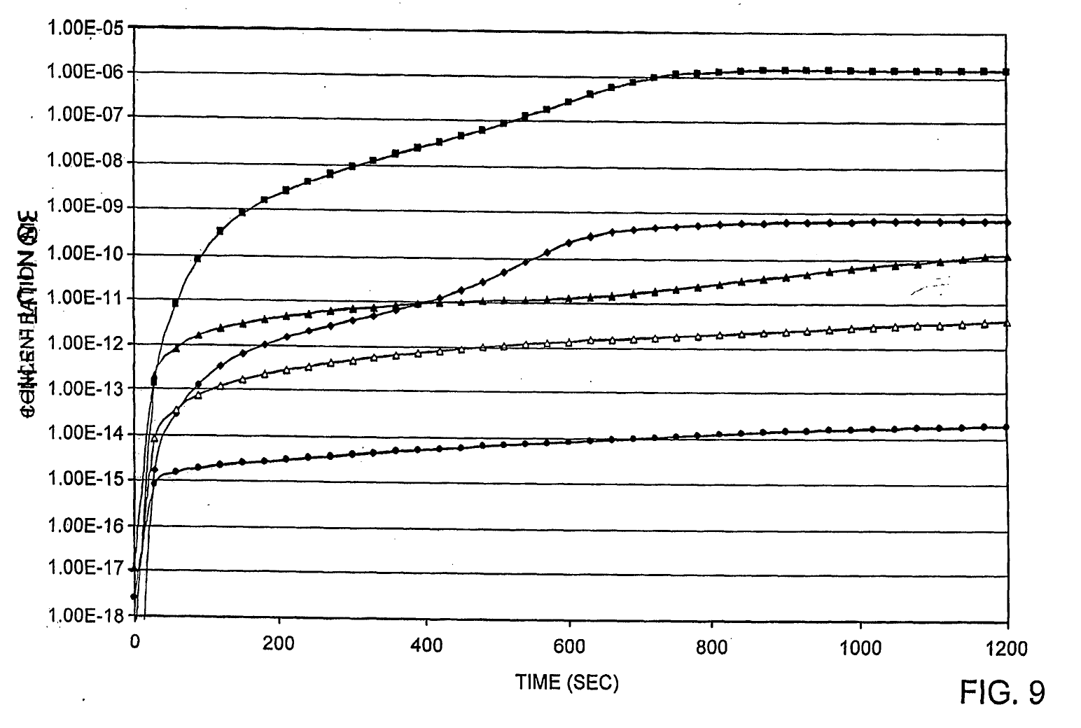

FIG. 9 represents the states and accumulation of the various serpin-AT-III complexes and the factor VIIIa dissociation products associated with the reaction termination during the course of the process. Factor VIIIa-A₂ domain dissociation and accumulation (filled diamonds) is a major contributor to the demise of the efficacy and concentration of the intrinsic factor Xase. This dominance is illustrated by the relative contribution of AT-III to factor IXa inhibition. The product of this complex, factor IXa-AT-III (open triangles) is observed to be at much lower concentration than the concentration of the factor VIIIa-A₂ dissociation product (filled diamonds). A relatively modest contribution of AT-III combining with factor VIIa-TF (filled circles) illustrates the larger role of TFPI attenuating the concentration of this complex.

Table 4 below illustrates anticipated residual levels of thrombin, factor Xa, factor VIIa-TF and factor IXa which would exist at 1200 seconds in a closed system.

TABLE 4
Residual Reactants at 1200 seconds

| | RESIDUAL CONCENTRATION |
|---|---|
| REACTANT | (Molar) |
| Factor VIIa | 6.57 × 10⁻⁹ |
| Factor IXa | 6.95 × 10⁻¹² |
| Factor VIIIa | 1.46 × 10⁻¹¹ |
| Factor Xa | 1.23 × 10⁻¹⁰ |
| Thrombin | 5.2 × 10⁻¹⁰ |

Referring to Table 4, the explicit conclusion of the model is that some enzyme remains but this has not been verified by empirical observation, nor has it been tested in empirical experiments. The suggested residual levels of serpins available at the conclusion of the reaction may be significant even though they represent only tiny fractions of the amounts of zymogen consumed in the overall reaction. At the low concentrations predicted, they would escape detection in typical “wet” experiments designed to evaluate the efficacy of AT-III interaction with any of its serpin companions.

The present “plasma” model does not provide for regulation of factor Va in the decay of prothrombinase since blood, (Rand, et al., 1996, Blood 88, 3432-3445), plasma and this model do not include significant levels of thrombomodulin, an essential element of the dynamic protein C system that serves to deplete factor Va

### Example 7

### Model Validation

Few empirical experiments have been published which display the complexity of the reaction system described by the numerical model. The empirical model which most closely approximates the model's conditions is represented by a previous report from this laboratory which examined the influence of alterations of blood clotting proteins within the normal range of plasma concentration (i.e., 50-150% of the mean plasma value) on the generation of thrombin were examined (Butenas, et al., 1999, Blood. 94, 2169-2178). These studies utilized an in vitro reaction system conducted with purified components at saturating PCPS concentrations; however, in this empirical model TF and VIIa were preincubated prior to the reaction system, i.e. preformation of the factor VIIa-TF complex. In the empirical experiments, prothrombin and AT-III had the most influence on the total amount of thrombin formed.

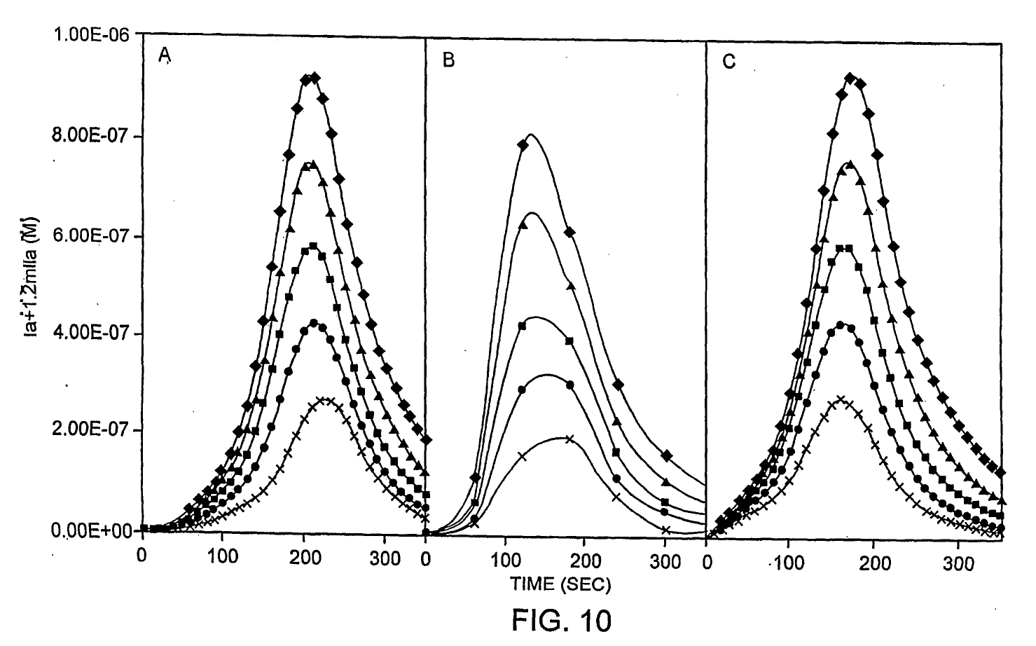

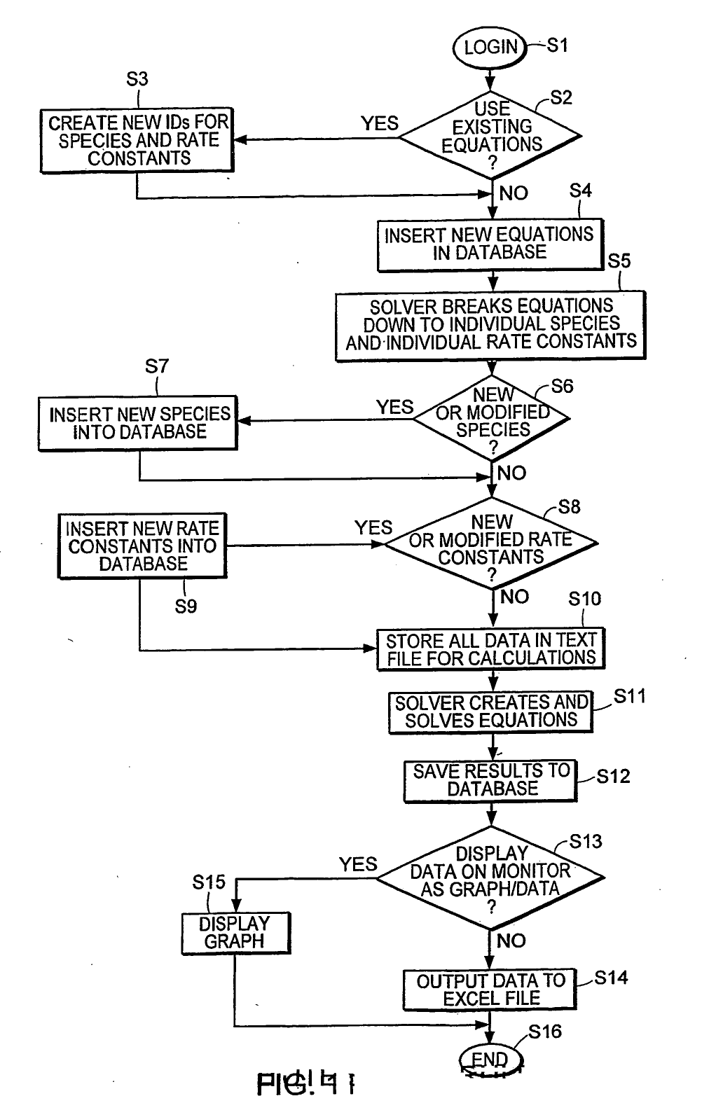

The numerical model was therefore computed with a factor VIIa-TF-preincubation term, which eliminates the formation of the extrintsic factor Xase. The empirical representation of the numerical and “wet” experiments exploring the influence of prothrombin concentration are presented in FIGS. 10A and 10B, which show the relative amounts of active thrombin produced as a function of time when prothrombin concentration is varied from 0.7 to 2.1 μM (i.e. 50-150% of the mean plasma value). The comparison of the empirical (FIG. 10B) and numerical (FIG. 10A) representations display great similarity in the relative amounts of thrombin produced for each experimental condition with similar peak values of thrombin observed. The major nonconformity of the numerical analysis with the empirical experiment is in the duration of the initiation phase observed in the empirical experiment which is noticeably shorter than that observed in the numerical analysis. A likely cause of this discrepancy is illustrated in FIG. 10C. The simulation in FIG. 10C is identical to the simulation in FIG. 10A except that it assumes the presence of 1% factor Va contamination in the factor V used in the empirical system experiment. With the assumption of 1% factor Va contamination in the experimental system, the numerical and empirical experiments are nearly identical (compare FIGS. 10A and 10C). The presence of a 1% contamination Va in human factor V preparations is highly likely based upon previous experiments and experience with natural preparations of this difficult molecule (Foster, et al., 1983 J. Biol. Chem. 258, 5608-5618; Nesheim, et al., 1979, J. Biol. Chem. 254, 508-517; Bloom, et al., 1979, Biochemistry 18,4419-4425; Katzmann, et al., 1981, Proc. Natl. Acad. Sci. USA 78, 162-166).

In a similar experiment, AT-III was varied over the same relative range. The results obtained were again similar to those observed in empirical “wet” experiments.

The present disclosure provides a predictive in silico model that can be used to describe and manipulate the formation of thrombin and other products by the TF induced coagulation pathway. In preferred invention examples, the complex reaction pathway of thrombin formation/inhibition observed in “wet” empirical analyses is approximated by the theoretical curves generated by our computer model. Most unique qualities of the thrombin producing reactions, including the observation of reaction thresholds governed by combinations of TFPI and AT-III, are represented in simulations generated by this model. In addition, the quantitative influence of alterations of coagulation protein concentrations within the normal range which influence empirical experimental outcomes are represented in a quantitatively respectable fashion by thrombin generation as predicted by the theoretical model.

Significantly, the invention provides a useful framework for the design and execution of experimental protocols involving this complex array of reagents and reactants. The evaluation of complex reaction arrays using intuition can be extraordinarily misleading in the anticipation of the influence of qualitative or quantitative alterations in individual constituents or reactions on a reaction system outcome. Also of central importance is the utility of numerical models in predicting presently inaccessible quantitative parameters whose required existence is anticipated and assured by the ultimate presence of catalysts, cofactors, serine proteases and their inhibitor complexes which must exist to give rise to the responses observed. The computer model has the capacity to anticipate the presence of minute concentrations of reactants and enzymes that must be present from estimation of the measurable products of their activation. The diminishingly small concentrations that must exist are frequently beyond the realm of the quantitative limits of current analytical devices and technologies. A prime example of this is the anticipated concentration of the factor VIIIa-factor IXa complex illustrated in FIG. 6. In order to produce the amounts of factor Xa and its complex with factor Va required to generate the thrombin concentrations which have been empirically measured, factor VIIIa-factor IXa complex concentrations between 10⁻¹⁸ and 10⁻¹⁴M must have been formed.

Comparisons of reaction profiles predicted to occur following preincubation of factor VIIa and, TF-PCPS to those predicted without mixing the initial factor VIIa and TF solutions are extraordinary when viewed from a kinetic perspective. At concentrations deemed biologically relevant the association rate constant (Table 2, k₄) between factor VIIa and TF clearly plays a significant role in the onset of this reaction. Empirical studies have illustrated the essential step of the activation of factor V by thrombin (Table 1, #16). Although the rate of prothrombin activation by factor Xa•membrane (Table 1, #9) is approximately 0.0001 that for prothrombinase, the model predicts that this tiny level of direct prothrombin activation is sufficient to provide the necessary thrombin initially required to catalyze the initial activations of factor V and factor VIII. The influence of factor VIIIa inactivation by A₂ fragment dissociation (Table 1, # 13, 15) and the significance of the regulatory influence of this process are also clearly manifest. Similarly, the relatively slow association between the factor VIIa-TF complex and AT-III might be presumed to be irrelevant but, as illustrated by the model, this inhibitor has significant influence on the ultimate propagation of the reaction.

In contrast to prior efforts in the field, the present invention incorporates the stoichiometric inhibitors TFPI and AT-III and provides a reasonably quantitative description of the generation of thrombin and other products and the regulation of this reaction under conditions incorporating normal plasma concentrations of protein with saturating concentrations of membrane.

Abbreviations used: tissue factor pathway inhibitor, TFPI; antithrombin III, AT-III, tissue factor, TF; PCPS, phospholipid vesicles composed of 75% phosphatidyl choline and 25% phosphatidyl serine.

Although a specific embodiment of the present invention has been described in detail herein with reference to the accompanying drawings, it is to be understood that the invention is not limited to that precise embodiment, and that various changes and modifications may be effected therein by one skilled in the art without departing from the spirit and scope of the invention as defined in the appended claims.

All references are incorporated herein by reference in their entirety.

1. A software program embodied on a computer-readable medium for predicting the speed and efficacy of a blood-clotting agent

2. A computer program product comprising a computer usable medium having computer readable program code means embodied in said medium for causing an application program to execute on a computer with a database for storing data therein, said computer program product for predicting the speed and efficacy of a blood clotting agent, said computer readable program code means comprising:
a. a first computer readable program code means for causing said computer to enter data into said database from a user interface;
b. a second computer readable program, code means for causing said computer to enter chemical equations into said database according to a user's input,
c. a third computer readable program code means for causing said computer to compile, differential equations corresponding to said chemical equations;
d. a fourth computer readable program code means for causing said computer to solve said differential equations; and
e. a fifth computer readable program code means for causing said computer to display said results of said solution to said differential equations

3. A method for determining or monitoring blood coagulation in silico, the method comprising performing a series of computer executable functions that manipulate input data featuring at least one of blood coagulation formation, expression and propagation variables, the functions generating, as output, a thrombin concentration, wherein the amount of thrombin is taken to be indicative of the blood coagulation.

4. The method of claim 3, wherein the computer executable functions include at least one of the following variables: 1) TFPI mediated inactivation of TF•VIIa and its product complexes; 2) AT-III mediated inactivation of IIa, mIIa, factor VIIa, factor IXa, and factor Xa; 3) initial activation of factor V and factor VII by thrombin generated by factor Xa-membrane; 4) factor VIIIa dissociation/activity loss; 5) binding competition, and kinetic activation steps which exist between tissue factor (TF) and factors VII and VIIa, and 6) activation of factor VII by IIa, factor Xa, and factor IXa.

5. A method for screening candidate compounds for ability to prevent or treat blood clotting in silico, the method comprising pre-selecting a drug for capacity to modulate at least one blood factor, performing a series of computer executable functions that manipulate input data featuring at least one of blood coagulation formation, expression and propagation variables, the functions generating, as output, a thrombin concentration, wherein the amount of thrombin produced by the drug is taken to be indicative of a compound that prevents or treats the blood clotting.

6. The method of claim 5, wherein the computer executable functions include at least one of the following variables: 1) TFPI mediated inactivation of TF•VIIa and its product complexes; 2) AT-III mediated inactivation of IIa, mIIIa, factor VIIa, factor IXa, and factor Xa; 3) initial activation of factor V and factor VIII by thrombin generated by factor Xa-membrane, 4) factor VIIIa dissociation/activity loss; 5) binding competition, and kinetic activation steps which exist between tissue factor (TF) and factors VII and VIIa, and 6) activation of factor VII by IIa, factor Xa, and factor IXa.

7. A method for designing a strategy to modulate a blood coagulation pathway which comprises a plurality of reaction steps, the method comprising:
simulating the effect of a compound on a component of the pathway, and
identifying affected steps in the pathway.

8. The method according to claim 7, wherein the compound inhibits the activity of one or more components of the pathway.

9. The method according to claim 7, wherein the compound is an antithrombotic compound.

10. The method according to claim 7, wherein the compound is an antihemorrhagic compound.

11. The method according to claim 7, further comprising the step of identifying and/or synthesizing a compound which produces the effect.

12. A method for modeling a patient-specific pathway comprising a plurality of reaction steps, the method comprising:
determining the concentrations of a plurality of reactants in the patient which participate in the reaction steps, thereby generating input data;
performing a series of computer executable functions manipulating the data; and
simulating time-dependent concentration profiles for one or more of the reactants.

13. The method according to claim 12, wherein the patient has a congenital or acquired condition which affects blood clotting.

14. The method according to claim 12 or 13, further comprising the steps of:
simulating the effect of a compound on a component of the pathway, and
identifying affected steps in the pathway

15. A method for designing a compound with a selected pharmacological activity; comprising:
simulating, a molecular pathway comprising a plurality of reaction steps;
simulating a compound which interacts with one or more components of the pathway; and
identifying interaction parameters which cause a desired effect on one or more steps of the pathway, and
identifying compound features which provide the selected activity.

16. The method according to claim 15, further comprising the step of identifying a compound which comprises the features identified.
